(19) United States
(12) Patent Application Publication
(10) Pub. No.: US 2023/0028364 A1
(43) Pub. Date: Jan. 26, 2023

(54) Method For Determining the Presence of Intestinal Parasites

(71) Applicant: Mobidiag OY, Espoo (FI)

(72) Inventors: Juha Kirveskari, Espoo (FI); Pasi Piiparinen, Espoo (FI); Jari Hirvonen, Espoo (FI); Juha Saharinen, Espoo (FI)

(21) Appl. No.: 17/776,159

(22) PCT Filed: Nov. 16, 2020
(86) PCT No.: PCT/FI2020/050766

§ 371 Date: May 11, 2022

(30) Foreign Application Priority Data
Nov. 15, 2019 (FI) 20195975

(51) Int. Cl. C12Q 1/6893; C12Q 1/6895

(52) U.S. Cl. CPC C12Q 1/6893; C12Q 1/6895; C12Q 2600/16; C12Q 1/686

(57) ABSTRACT

This invention relates to the field of detection of intestinal parasites from patient, food or environmental samples, preferably from a stool sample. Particularly, the present invention provides a polymerase chain reaction (PCR) based assay method for detection of intestinal parasite infection, particularly the infection of parasite species selected from a group consisting of Hymenolepis nana, Hymenolepis diminuta, Fasciolopsis buski, Encephalitozoon spp. (such as E. intestinalis, E. cuniculi and E. hellem), Enterocytozoon bieneusi, Enterobius vermicularis, Diphyllobothrium latum, Diphyllobothrium nihonkaiense, Schistosoma mansoni, Blastocystis hominis, Ancylostoma duodenale and liver worms, such as Clonorchis sinensis, Opisthorchis spp., and Metorchis spp. The present invention further provides materials such as primers, primer pairs and probes for use in the method of the invention. Preferably, the method of the invention is a multiplex real-time PCR assay for rapid determination of clinically important intestinal parasites.

### FIELD OF THE INVENTION

This invention relates to the field of detection of intestinal parasites from patient, food or environmental samples, preferably from a stool sample. Particularly, the present invention provides a polymerase chain reaction (PCR) based assay method for detection of intestinal parasite infection, particularly the infection of parasite species selected from a group consisting of Hymenolepis nana, Hymenolepis diminuta, Fasciolopsis buski, Encephalitozoon spp. (such as E. intestinalis, E. cuniculi and E. hellem), Enterocytozoon bieneusi, Enterobius vermicularis, Diphyllobothrium latum, Diphyllobothrium nihonkaiense, Schistosoma mansoni, Blastocystis hominis, Ancylostoma duodenale and liver worms, such as Clonorchis sinensis, Opisthorchis spp., and Metorchis spp. The present invention further provides materials such as primers, primer pairs and probes for use in the method of the invention. Preferably, the method of the invention is a multiplex real-time PCR assay for rapid determination of clinically important intestinal parasites.

### BACKGROUND OF THE INVENTION

Intestinal parasite infections are a major health problem worldwide causing morbidity, but also mortality especially of infants in the developing countries. Intestinal parasite infections are also a reported problem for travelers and are commonly caused by contamination of food or water, infected soil and improper hygiene. Intestinal parasite infections may cause mild symptoms, but severe infections with abdominal pain, bloody diarrhoea and vomiting exist. These symptoms negatively impact nutritional status, loss of appetite, weight loss, and intestinal blood loss that can often result in anemia.

At present, there is no uniform standard for the identification of intestinal parasites (Garcia et al., 2018). At present, the diagnosis of many parasites depends on microscopy of a stool sample. It is well known that microscopy takes a long time, has low inspection efficiency, and requires high clinical experience for the examiner. Moreover, intestinal parasites may be present as cysts or eggs that are similar in shape with multiple parasite species and cannot thus be distinguished from each other by ordinary light microscope. The prior art methods thus often lead to misjudgments. Also immunodiagnosis have been used for the parasite detection, but although immunodiagnosis is more efficient than microscopy, it is often limited by sensitivity and specificity.

Although a number of PCR based assays for detecting intestinal parasite species are already disclosed, there is still a need in the field for further PCR assays which are able to provide high specificity and reliability for the detection of specific intestinal parasite species, for instance in multiplex assays. The present inventors have now located DNA sequence regions in parasite genomes that are surprisingly well-suited for specific and sensitive amplification of markers in particular intestinal parasite species.

The sample matrix, which in parasite diagnostics is commonly a stool or food sample, is likely to contain a host of PCR inhibitors. This reduces amplification efficiency of the PCR reaction and thus even more careful optimization is expected from the amplicon design step to verify that all templates and copy numbers are amplified equally but also efficiently enough. Hence, oligonucleotide design enabling high PCR efficiency (optimally as close to 100% as possible) is required. The detection method used may also affect amplification efficiency and/or bias.

The present inventors have now located DNA sequence regions that are well suited for specific and sensitive amplification and quantification of intestinal parasite species. The amplicons have been designed to be so specific that they can be combined into any multiplex sets with each other. Naturally a prerequisite to this is that all the disclosed amplicons have also been designed to amplify in the same reaction and cycling conditions. The aim of the invention is to replace antigen testing and microscoping as a screening test for intestinal parasites, and thus provide process improvements for the laboratory and clinical benefits in improved patient management by providing rapidly a rich set of information. Further, infection control could benefit if clinical microbiology laboratories could readily differentiate between intestinal parasite species.

### SUMMARY OF THE INVENTION

The number of intestinal parasites is large and a parasite test method should optimally identify as many as possible. Having one PCR reaction per species can be cumbersome, since the number of samples tested is typically large. It would be optimal to detect multiple species within one reaction. In a PCR setting the most obvious alternative is ‘multiplex’ PCR amplification. In multiplex PCR, several oligonucleotide sets, each designed to amplify one species/species group, are included in the same reaction vessel and each oligonucleotide set is used to amplify its respective pathogen DNA during the same PCR reaction. In this invention, we describe a PCR based method for rapid detection of clinically important intestinal parasites, particularly Hymenolepis nana, Hymenolepis diminuta, Fasciolopsis buski, Encephalitozoon spp. (such as E. intestinalis, E. cuniculi and E. hellem), Enterocytozoon bieneusi, Enterobius vermicularis, Diphyllobothrium latum, Diphyllobothrium nihonkaiense, Schistosoma mansoni, Blastocystis hominis, Ancylostoma duodenale and liver worms, such as Clonorchis sinensis, Opisthorchis spp., and Metorchis spp. The present invention discloses primers and probes designed for target sequences conserved in said intestinal parasites. These primers and probes are compatible for use in any multiplex qPCR determining the presence of multiple intestinal parasites.

Multiplex PCR presents a challenge for quantitation of the pathogen DNA (qPCR): the different amplicons compete for the same PCR reaction components (eg. DNA polymerase and MgCl2) and this can compromise the quantitative nature of the reaction between and, especially, quantitative comparisons between samples. It is commonly known in the art that there is bias in the amplification efficiencies between different template amounts or lengths so that e.g. short amplicons are favoured in the expense of longer ones.

At the same time, undesired cross-reactions of multiplex set oligo combinations must be avoided. One must also remember to check mis-priming to any other sequences present in the sample.

Finding suitable primer and probe sequences for the detection of a diverse group of pathogenic microbes can be far from trivial especially when designing multiplex set ups where all amplicons and templates should be amplified with equal efficiency. Many of the species are relatively closely related, making it challenging to locate sequences that are unique for each species. Some genes possess complex repetive closely related elements which is challenging from the amplicon design point of view, especially when designing amplicons for multiplex PCR.

The sample matrix, which in intestinal parasite diagnostics is commonly a stool or food sample, is likely to contain a host of PCR inhibitors. This reduces amplification efficiency of the PCR reaction and thus even more careful optimization is expected from the amplicon design step to verify that all templates and copy numbers are amplified equally but also efficiently enough. Hence, oligonucleotide design enabling high PCR efficiency (optimally as close to 100% as possible) is required. The detection method used may also affect amplification efficiency and/or bias.

In an aspect, the present invention is directed to a method for determining the presence of one or more intestinal parasites in a biological sample comprising the steps of:

i) contacting the sample or nucleic acid isolated therefrom with oligonucleotide primers in an amplification assay to provide a reaction mix for nucleic acid amplification;

ii) performing a nucleic acid amplification reaction with the reaction mix obtained from step i) comprising DNA from the biological sample as a template, so that the target sequences of the intestinal parasite(s) is/are specifically amplified, whenever said sequences are present in the sample; and

iii) detecting the presence of an amplified target sequence in the reaction mix, wherein the presence of the target sequence is indicative of the presence of intestinal parasites in the sample;

wherein said one or more intestinal parasites is/are selected from the group consisting of Hymenolepis nana, Hymenolepis diminuta, Fasciolopsis buski, Encephalitozoon spp. (such as E. intestinalis, E. cuniculi and E. hellem), Enterocytozoon bieneusi, Enterobius vermicularis, Diphyllobothrium latum, Diphyllobothrium nihonkaiense, Schistosoma mansoni, Blastocystis hominis, Ancylostoma duodenale and liver worms, such as Clonorchis sinensis, Opisthorchis spp., and Metorchis spp;

wherein said target sequence(s) is/are selected from the group consisting of the sequences as defined by SEQ ID Nos: 1-16 and 46-47, wherein said oligonucleotide primers comprise a primer pair which binds to one of the target sequences as defined by SEQ ID Nos: 1-16 and 46-47 and allow amplification of at least part of the target sequence in step ii).

### BRIEF DESCRIPTION OF THE DRAWINGS

FIG. 1. Amplification curve for a PCR reaction with primers for Hymenolepis nana and Hymenolepis diminuta.

FIG. 2. Amplification curve for a PCR reaction with primers for Fasciolopsis buski.

FIG. 3. Amplification curve for a PCR reaction with primers for A. E. cuniculi and B. E. intestinalis.

FIG. 4. Amplification curve for a PCR reaction with primers for A. E. hellem and B. Enterocytozoon bieneusi.

FIG. 5. Amplification curve for a PCR reaction with primers for A. Enterobius vermicularis and B. Diphyllobothrium spp.

FIG. 6. Amplification curve for a PCR reaction with primers for A. Diphyllobothrium nihonkaise and B. Diphyllobothrium nihonkaise/klebanovskii.

FIG. 7. Amplification curve for a PCR reaction with primers for A. Schistosoma mansoni, and B. Blastocystis hominis.

FIG. 8. Amplification curve for a PCR reaction with primers for liver worms Clonorchis sinensis, Opisthorchis spp., and Metorchis spp.

FIG. 9. Preferred PCR protocol for the present invention.

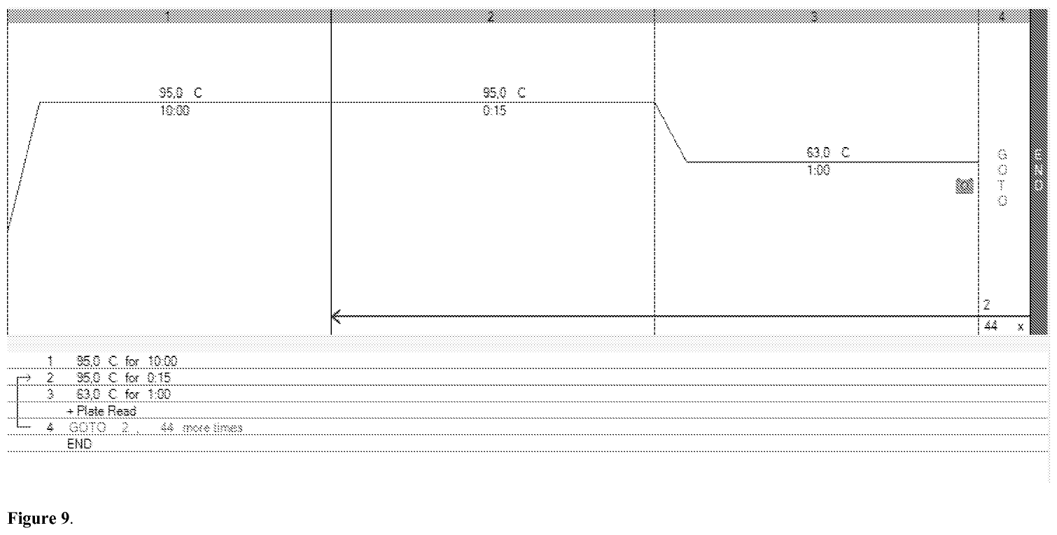

### DETAILED DESCRIPTION OF THE EMBODIMENTS

The present invention provides a nucleic acid amplification based assay method for detection of intestinal parasites, particularly one or more intestinal parasites selected from the group consisting of Hymenolepis nana, Hymenolepis diminuta, Fasciolopsis buski, Encephalitozoon spp. (such as E. intestinalis, E. cuniculi and E. hellem), Enterocytozoon bieneusi, Enterobius vermicularis, Diphyllobothrium latum, Diphyllobothrium nihonkaiense, Schistosoma mansoni, Blastocystis hominis, Ancylostoma duodenale and liver worms, such as Clonorchis sinensis, Opisthorchis spp., and Metorchis spp. The present invention further provides materials such as primers, primer pairs (i.e. a pair of a forward primer and a reverse primer) and probes for use in the method of the invention. Particularly, the present invention provides a method for determining the presence of intestinal parasites in a biological sample comprising the steps of i) contacting the sample or nucleic acid isolated therefrom with oligonucleotide primers in an amplification assay to provide a reaction mix for nucleic acid amplification;

ii) performing a nucleic acid amplification reaction with the reaction mix obtained from step i) comprising DNA from the biological sample as a template, so that the target sequences of the intestinal parasite(s) is/are specifically amplified, whenever said sequences are present in the sample; and

iii) detecting the presence of an amplified target sequence in the reaction mix, wherein the presence of the target sequence is indicative of the presence of intestinal parasites in the sample;

wherein said target sequence(s) is/are selected from the group consisting of the sequences as defined by SEQ ID Nos: 1-16 and 46-47, wherein said oligonucleotide primers comprise a primer pair which binds to one of the target sequences as defined by SEQ ID Nos: 1-16 and 46-47 and allow amplification of at least part of the target sequence in step ii).

Said biological sample can be a stool sample, a food sample, such as a meat sample, or any environmental sample. The sample may be enriched before step i).

Preferably, said nucleic acid amplification reaction is a polymerase chain reaction (PCR). As well-known in the art, PCR is a method whereby a limited segment of a nucleic acid molecule, i.e. a target sequence, is amplified repetitively to produce a large amount of DNA molecules consisting of only that segment. The procedure depends on repetition of a large number of priming and transcription cycles. In each cycle, two oligonucleotide primers, i.e. a forward primer and a reverse primer, bind to the segment, and define the limits of the segment. A primer-dependent DNA polymerase then transcribes, or replicates, the strands to which the primers have bound. The resulting PCR products are called amplicons. In a particular example, the methods disclosed herein include the step of PCR amplifying a portion of the genome of an intestinal parasite.

“Target sequence” as defined herein is a nucleic acid segment present in the genome of a intestinal parasite whose detection, quantitation, qualitative detection, or a combination thereof, is intended. For example, the target sequence is a specific nucleic acid in intestinal parasite genome, the amplification of which is intended. Purification or isolation of a template molecule, if needed, for initiation of the amplification reaction can be conducted by methods known to those in the art. For example, isolation of the template can be achieved by using a commercially available purification kit or the like.

Preferred target sequences (or amplicons) amplified in target organisms are listed in Table 1. However, a person skilled in the art knows that these target sequences naturally vary in related strains. This minor variation can be taken into account while designing primers suitable to amplify said amplicons in the method of the present invention. Preferably, at least 20, 25, 30, 35, 40, 50, 60, 70, 80, 90 100, or 125 nucleotides long sequence of each of the target sequences selected from the group consisting of SEQ ID NOS:1-16 and 46-47 are amplified in the method.

TABLE 1
Target sequences (5′→3′) amplified in target organisms. Only one strand of each
nucleic acid sequence is shown, but the complementary strand is understood as included
by any reference to the displayed strand.

Hymenolepis cox1
AATTCCTGATGCTTTTGGGTTTTATGGTTTATTATTTGCTATGTTTTCTATAGTGTGCTTA
GGTTGTAGTGTGTGGGCTCATCATATGTTTACTGTTGGTTTGGATGTTAAGACGGCTGTAT
TTT (SEQ ID NO: 1)
Hymenolepis cox1, v2
AATTCCTGATGCTTTTGGGTTTTATGGGCTCTTGTTTGCCATGTTTTCTATTGTTTGTTTA
GGTAGAAGTGTTTGAGGGCATCATATGTTTACTGTTGGTTTAGATGTAAAGACGGCAGTGT
TOT (SEQ ID NO: 2)
Fasciolopsis buski ITS1
CACTGTTCAAGTGGTATTGATTGGGTTCGCCCATTCTTTGCCATTGCCCTCGCATGCACCT
GGTCCTTGTGGCCGGACTGCACGTACGTCGCCCGGCGGTGCCTATCCCGGGTAGGACTGAT
AACCTGG (SEQ ID NO: 3)
Encephalitozoon sp 18S
GACGAAGATCGGAAGGTCTGAGTCCTGAGTGTTAGATAAGATATAAGTCGTAACATGGCTG
CTGTTGGAGAACCAGCAGCAGGATCAGTATGTTGTTGTGTTTTGATGGATGTTTGTTTGTT
TGTTTGTGGTTTCTCTGTTCACGGGATTGATTGGCATTAGCG (SEQ ID NO: 4)
Encephalitozoon sp 18S v2
GACGAAGATTGGAAGGTCTGAGTCCTTCGTGTTAGATAAGATATAAGTCGTAACGCGGCTG
CTGTTGGAGAACCAGCAGCAGGATCAGTATTTGAGAGATTGGGGGGAATTTTTTTGATTTG
AGGATCCACGGGATTGATAGGCATTAGCA (SEQ ID NO: 5)
Encephalitozoon sp 18S v3
GACGAAGATTGAGAGGTCTGAGTCTTTCGTGTTAGATAAGATATAAGTCGTAACATGGCTG
CTGTTGGAGAACCAGCAGCAGGATCAGTATGTTGATTTGATTGATTTGTGGGGATTTTTAG
TTTTTTAGTTTTTCTTTCTCTATCCATGGGATTGATTGGCATTATCT (SEQ ID NO: 6)
Enterocytozoon bieneusi 18S
GAGTGTAGTATAGACTGGCGAAGAATGAAATCTCAAGACCCAGTTTGGACTAACGGAGGCG
AAGGCGACACTCTTAGACGTATCTTAGGATCAAGGACGA (SEQ ID NO: 7)
Enterobius vermicularis ITS
GCAGAGCTTTTCCAAAATTTATTTCCAAGCCACAGACTCACTGATGTTCATGTCTGAGCCGGAACG
AGAAATTACCTCAAACTTGGG (SEQ ID NO: 8)
Diphyllobothrium latum/nihonkaiense cox1
CCAGTTATTACAGGTGTGAGATTGAATAAGTATTTATTACAATGTCATTGTATAGTTTCTA
ATGTTGGTTTCAATTTATGTTTTTTCCCTATGCATTACTTTGGTGTGTGCGGTTTACCACG
TCGTGTGTGTGTGTACGAGTCGGGTTATGCTTGA (SEQ ID NO: 9)
Diphyllobothrium latum/nihonkaiense cox1 v2
CCAGTTATTACTGGTGTAAGATTGAATAAGTATTTACTACAATGTCATTGTATAGTTTCTA
ATGTTGGTTTCAATTTATGTTTTTTTCCCATGCATTATTTTGGTGTGTGCGGTTTACCACG
TCGTGTGTGCGTATATGAGTCAGGTTATGCTTGA (SEQ ID NO: 10)
Diphyllobothrium latum/nihonkaiense cox1 v3
CCAGTTATTACTGGTGTAAGATTGAATAAGTATTTACTACAATGTCATTGTATAGTTTCTA
ATGTTGGTTTCAATTTATGTTTTTTTCCTATGCATTATTTTGGTGTGTGCGGTTTACCACG
TCGTGTGTGTGTATATGAGTCAGGTTATGCTTGA (SEQ ID NO: 11)
Diphyllobothrium latum/nihonkaiense cox1 v4
CCAGTTATTACTGGTGTGAGATTGAATAAGTATTTACTACAATGTCATTGTATAGTTTCTA
ATGTTGGTTTCAATTTATGTTTTTTTCCTATGCATTATTTTGGTGTGTGCGGTTTACCACG
TCGTGTGTGCGTATATGAGTCAGGTTATGCTTGA (SEQ ID NO: 12)
Schistosoma mansoni cox1
AGGTGTTTTCATGACTTTATATGTTGAATAGTTGCGGTATGCGGGTTTTAGATCCCATAGT
ATGGTGATTAGTCGGTTTTATATTTTTATTTACGGTTGGTGGTGTCACAGGGGTGGCTTTA
TCTGCATCTGCT (SEQ ID NO: 13)
Blastocystis hominis 18S
TCAGCTTTCGATGGTAGTGTATTGGACTACCATGGCAGTAACGGGTAACGAAGAATTTGGG
TTCGATTTCGGAGAGGGAGCC (SEQ ID NO: 14)
Blastocystis hominis 18S v2
TCAGCTTTCGATGGTAGTATATGGGCCTACCATGGCAGTAACGGGTAACGAAGAATTTGGG
TTCGATTTCGGAGAGGGAGCC (SEQ ID NO: 15)
C.sinensis/Opisthorchis sp./Metorchis sp. 18S
AGCTCGTAGTTGGATCTGGGTCGCATGGCTACATGCCGTTGCTCGTATTCCTGGCCTGGTT
CACACCGGGACGGGTTTGTGAGTCGGTGTCGTGG (SEQ ID NO: 16)
Ancylostoma duodenale ITS gB 1
CCCATGAGACATACAAAAAGGTAATGCCGCCGTCTGGTTCAGGGTTGTTTATATCTACTAC
AGTGTAGCTTGTGGCACTGTTTGTCGAACGGCACTTGCTTTTAGCGATTCCCGTTCTAGAT
CAGAATATATTGCAACATGTACGTTAGCTGGCTAGTTTGCTAACGTGCGCTGAATGACAGC
AAACTCGTTGTTGCTGCTGAATCGTTCACCGACTTTAGAACGTTTCGGGTCTCGACTATAC
GCCCGTTTTCGGATC (SEQ ID NO: 46)
Ancylostoma duodenale ITS gB 2
CCCATGAGACATACAAAAAGGTAATGCCGCCTATATCTACTACAGTGCAGCTTGTGGCACT
GTTTGTCGAACGGCACTTGCTTTTAGCGATTCCCGTTCTAGATCAGAATATATTGCAACAT
GTACGTTGGCTGGCTAGTTTGCTAACGTGCGCTGAATGACAGCAAACTCGTTGTTGCTGCT
GAATCGTTTACCGACTTTAGAACGTTTCGGGTCTCGACTATACGCCCGTTTTCGGATC
(SEQ ID NO: 47)

Primer pairs, which are preferably used in the present method to amplify the target sequences are listed in Table 2.

TABLE 2
Examples of primer sequences (5′→3′) for amplification of the target sequences
listed in Table 1.

Primer pair A), Hymenolepis cox1
forward primer: AATTCCTGATGCTTTTGGGTTTTATG (SEQ ID NO: 17)
reverse primer: AGAACACTGCCGTCTTTACATCTAA (SEQ ID NO: 18)
Primer pair B), Hymenolepis cox1, v2
forward primer: AATTCCTGATGCTTTTGGGTTTTATG (SEQ ID NO: 17)
reverse primer: AAATACAGCCGTCTTAACATCCAA (SEQ ID NO: 19)
Primer pair C), Fasciolopsis buski ITS1
forward primer: CACTGTTCAAGTGGTATTGATTG (SEQ ID NO: 20)
reverse primer: CCAGGTTATCAGTCCTACCC (SEQ ID NO: 21)
Primer pair D), Encephalitozoon sp 18S
forward primer: CTGAGTCCTGAGTGTTAGATAAGA (SEQ ID NO: 22)
reverse primer: CTAATGCCAATCAATCCCGTG (SEQ ID NO: 23)
Primer pair E), Encephalitozoon sp 18S v2
forward primer: GTCCTTCGTGTTAGATAAGATATAAGTC (SEQ ID NO: 24)
reverse primer: AGATAATGCCAATCAATCCCATG (SEQ ID NO: 25)
Primer pair F), Encephalitozoon sp 18S v3
forward primer: GACGAAGATTGAGAGGTCTGA (SEQ ID NO: 26)
reverse primer: CTAATGCCTATCAATCCCGTG (SEQ ID NO: 27)
Primer pair G), Enterocytozoon bieneusi 18S
forward primer: GAGTGTAGTATAGACTGGCGAA (SEQ ID NO: 28)
reverse primer: TCGTCCTTGATCCTAAGATACG (SEQ ID NO: 29)
Primer pair H), Enterobius vermicularis ITS
forward primer: GCAGAGCTTTTCCAAAATTTATTTCC (SEQ ID NO: 30)
reverse primer: CCCAAGTTTGAGGTAATTTCTCG (SEQ ID NO: 31)
Primer pair I), Diphyllobothrium latum/nihonkaiense cox1
forward primer: CCAGTTATTACTGGTGTAAGATTGAA (SEQ ID NO: 32
reverse primer: TCAAGCATAACCTGACTCATATAC(SEQ ID NO: 33)
Primer pair J), Diphyllobothrium latum/nihonkaiense cox1 v2
forward primer: CCAGTTATTACTGGTGTAAGATTGAA (SEQ ID NO: 34
reverse primer: TCAAGCATAACCTGACTCATATAC(SEQ ID NO: 35)
Primer pair K), Diphyllobothrium latum/nihonkaiense cox1 v3
forward primer: CCAGTTATTACAGGTGTGAGATTG (SEQ ID NO: 36)
reverse primer: CAAGCATAACCCGACTCGTA (SEQ ID NO: 37)
Primer pair L), Diphyllobothrium latum/nihonkaiense cox1 v4
forward primer: CCAGTTATTACAGGTGTGAGATTG (SEQ ID NO: 36)
reverse primer: CAAGCATAACCCGACTCGTA (SEQ ID NO: 37)
Primer pair M), Schistosoma mansoni coxl
forward primer: AGGTGTTTTCATGACTTTATATGTTGA (SEQ ID NO: 38
reverse primer: AGCAGATGCAGATAAAGCCA (SEQ ID NO: 39)
Primer pair N), Blastocystis hominis 18S
forward primer: CAGCTTTCGATGGTAGTGTATTG (SEQ ID NO: 40)
reverse primer: GGCTCCCTCTCCGAAATC (SEQ ID NO: 41)
Primer pair O), Blastocystis hominis 18S v2
forward primer: TCAGCTTTCGATGGTAGTATATGG (SEQ ID NO: 42)
reverse primer: GGCTCCCTCTCCGAAATC (SEQ ID NO: 43)
Primer pair P), C.sinensis/Opisthorchis sp./Metorchis sp. 18S
forward primer: AGCTCGTAGTTGGATCTGG (SEQ ID NO: 44)
reverse primer: CCACCAATCATGCTAACACC (SEQ ID NO: 45)
Primer pair Q), Ancylostoma duodenale ITS.3.1
forward primer: CAGTGTAGCTTGTGGCAC (SEQ ID NO: 48)
reverse primer: CAGCTAACGTACATGTTGCAATA (SEQ ID NO: 49)
Primer pair R), Ancylostoma duodenale ITS.3.2
forward primer: ACAGTGCAGCTTGTGGCA (SEQ ID NO: 50)
reverse primer: CAGCCAACGTACATGTTGCAATA (SEQ ID NO: 51)

The method of the invention is characterized in that the presence of the amplified target sequence, i.e. the product, of each of primer pairs in the PCR reaction in step iv) indicates the presence of intestinal parasites in the sample in the following way:
the product of primer pair A) or B) indicates the presence of Hymenolepis nana or Hymenolepis diminuta;
the product of primer pair C) indicates the presence of Fasciolopsis buski;
the product of primer pair D), E) or F) indicates the presence of E. intestinalis, E. cuniculi or E. hellem;
the product of primer pair G) indicates the presence of Enterocytozoon bieneusi;
the product of primer pair H) indicates the presence of Enterobius vermicularis;
the product of primer pair I), J), K), or L) indicates the presence of Diphyllobothrium latum, or Diphyllobothrium nihonkaiense;
the product of primer pair M) indicates the presence of Schistosoma mansoni;
the product of primer pair N) or O) indicates the presence of Blastocystis hominis;
the product of primer pair P) indicates the presence of liver worms, such as Clonorchis sinensis, Opisthorchis spp., and Metorchis spp.; and
the product of primer pair Q) or R) indicates the presence of Ancylostoma duodenale.

Preferably, each primer of said primer pairs is less than 25, 30, 35, 40, 45, 50 or 55 nucleotides long, and more preferably, less than 50 nucleotides long. Each of the present primers can also be defined as comprising or consisting of at least 10, 15, 16, 17 or 18 contiguous nucleotides present in at least one primer sequence selected from the group consisting of SEQ ID NOS:17-45 and 48-51. Each of the present primers can further be defined as having at least 80%, 85%, or 90% sequence identity to at least one primer sequence selected from the group consisting of SEQ ID NOS:17-45 and 48-51.

One specific embodiment of the invention is to perform said method as a real-time polymerase chain reaction and in that case nucleic acid probes comprising or consisting of the following sequences are specifically used with each of primer pairs A) to T) in the following manner:

-the probe for primer pair A):
(SEQ ID NO: 52)
5′-AGTGTGCTTAGGTTGTAGTGTGTGGGCTCATC-3′
-the probe for primer pair B):
(SEQ ID NO: 53)
5′-TGTTTGCCATGTTTTCTATTGTTTGTTTAGG-3′
-the probe for primer pair C):
(SEQ ID NO: 54)
5′-TTCGCCCATTCTTTGCCATTGCCC-3′
-the probe for primer pair D):
(SEQ ID NO: 55)
5′-CTGATCCTGCTGCTGGTTCTCCAACAG-3′
-the probe for primer pair E):
(SEQ ID NO: 56)
5′-ATGATCCTGCTAATGGTTCTCCAACAGCA-3′
-the probe for primer pair F):
(SEQ ID NO: 57)
5′-ATGATCCTGCTAATGGTTCTCCAACAGCA-3′
-the probe for primer pair G):
(SEQ ID NO: 58)
5′-AGTGTCGCCTTCGCCTCCGTTAG-3′
-the probe for primer pair H):
(SEQ ID NO: 59)
5′-TCCGGCTCAGACATGAACATCAGTGAGTCT-3′
-the probe for primer pair I):
(SEQ ID NO: 60)
5′-ACACGACGTGGTAAACCGCACACA-3′
-the probe for primer pair J):
(SEQ ID NO: 61)
5′-ACACGACGTGGTAAACCGCACACA-3′
-the probe for primer pair K):
(SEQ ID NO: 62)
5′-ACACGACGTGGTAAACCGCACACA-3′
-the probe for primer pair L):
(SEQ ID NO: 63)
5′-ACACGACGTGGTAAACCGCACACA-3′
-the probe for primer pair M):
(SEQ ID NO: 64)
5′-CCCCTGTGACACCACCAACCGT-3′
-the probe for primer pair N):
(SEQ ID NO: 65)
5′-AAATTCTTCGTTACCCGTTACTGCCATGGT-3′
-the probe for primer pair O):
(SEQ ID NO: 66)
5′-AAATTCTTCGTTACCCGTTACTGCCATGGT-3′
-the probe for primer pair P):
(SEQ ID NO: 67)
5′-TTGCTCGTATTCCTGGCCTGGTTCA-3′

The melting temperature, Tm, of some of the probes (such as probes for primer pairs G), H), K) and L)) is preferably increased at least 5 degrees ° C. by addition of modified nucleotides. The amount of modified nucleotides in one probe is 1, 2, 3 or preferably 4. The underlined nucleotides in the above list are modified nucleotides each increasing the Tm of the probe. The modified nucleotide can be a LNA nucleotide (Exiqon A/S), minor groove binder (MGB™), SuperBase, or Peptide Nucleic Acid (PNA) or any other modification increasing the Tm of the probe.

Preferably, the above probes comprise the sequences as defined and are less than 25, 30, 35, 40, 45, 50 or 55 nucleotides long, and more preferably, less than 50 nucleotides long. Each of the present probes can also be defined as comprising or consisting of at least 10 or 15, 16, 17 or 18 contiguous nucleotides present in one probe sequence selected from the group consisting of SEQ ID NOS:52-67 or complements thereof.

A probe preferably includes a detectable label, such as a fluorophore. Examples of the fluorophores are fluorescein and derivatives thereof such as 6-carboxyfluorescein (FAM) and fluorescein isothiocyanate (FITC). The detectable label may produce a signal in the presence of a target amplicon, or result in a decreased signal in the presence of a target amplicon, depending on the particular construction of the probe.

The method of the invention is based on multiplex PCR technique simultaneously analyzing nucleic acids of many templates from a sample, i.e. a multiplex PCR reaction comprises a set of primer pairs capable of simultaneous amplification of various target sequences.

In a further embodiment, the invention provides nucleotide probes comprising or consisting of any of the probe sequences as defined above.

The present invention is preferably directed to a method for determining the presence of intestinal parasites in a sample, wherein the presence of at least one of the pathogens Hymenolepis nana, Hymenolepis diminuta, Fasciolopsis buski, Encephalitozoon spp. (such as E. intestinalis, E. cuniculi and E. hellem), Enterocytozoon bieneusi, Enterobius vermicularis, Diphyllobothrium latum, Diphyllobothrium nihonkaiense, Schistosoma mansoni, Blastocystis hominis, Ancylostoma duodenale and liver worms, such as Clonorchis sinensis, Opisthorchis spp., and Metorchis spp., is detected. In preferred embodiments, the presence of any combination of the above listed intestinal parasites is detected. Accordingly, each combination of 2, 3, 4, 5, 6, 7, 8 or more of said intestinal parasites is a preferred embodiment for the present invention.

In a preferred embodiment, at least the presence of Hymenolepis nana and Hymenolepis diminuta are detected in the method, wherein the target sequences are at least as defined by SEQ ID NOS: 1 and 2. More preferably, a primer pair set allowing amplification of at least part of said target sequences comprise or consist of at least 15 consecutive nucleotides of the sequences as defined in SEQ ID NOS: 17, 18, and 19.

In a preferred embodiment, at least the presence of Fasciolopsis buski is detected in the method, wherein the target sequence is at least as defined by SEQ ID NO: 3. More preferably, a primer pair set allowing amplification of at least part of said target sequences comprise or consist of at least 15 consecutive nucleotides of the sequences as defined in SEQ ID NOS: 20 and 21.

In a preferred embodiment, at least the presence of Encephalitozoon spp. (such as E. intestinalis, E. cuniculi and E. hellem) are detected in the method, wherein the target sequences are at least as defined by SEQ ID NOS:4-6. More preferably, a primer pair set allowing amplification of at least part of said target sequences comprise or consist of at least 15 consecutive nucleotides of the sequences as defined in SEQ ID NOS:22-27.

In a preferred embodiment, at least the presence of Enterocytozoon bieneusi is detected in the method, wherein the target sequence is at least as defined by SEQ ID NO:7. More preferably, a primer pair set allowing amplification of at least part of said target sequences comprise or consist of at least 15 consecutive nucleotides of the sequences as defined in SEQ ID NOS: 28 and 29.

In a preferred embodiment, at least the presence of Enterobius vermicularis is detected in the method, wherein the target sequence is at least as defined by SEQ ID NO:8. More preferably, a primer pair set allowing amplification of at least part of said target sequences comprise or consist of at least 15 consecutive nucleotides of the sequences as defined in SEQ ID NOS: 30 and 31.

In a preferred embodiment, at least the presence of Diphyllobothrium latum and Diphyllobothrium nihonkaiense are detected in the method, wherein the target sequences are at least as defined by SEQ ID NOS:9-12. More preferably, a primer pair set allowing amplification of at least part of said target sequences comprise or consist of at least 15 consecutive nucleotides of the sequences as defined in SEQ ID NOS:32-37.

In a preferred embodiment, at least the presence of Schistosoma mansoni is detected in the method, wherein the target sequence is at least as defined by SEQ ID NO:13. More preferably, a primer pair set allowing amplification of at least part of said target sequences comprise or consist of at least 15 consecutive nucleotides of the sequences as defined in SEQ ID NOS:38 and 39.

In a preferred embodiment, at least the presence of Blastocystis hominis is detected in the method, wherein the target sequences are at least as defined by SEQ ID NOS:14 and 15. More preferably, a primer pair set allowing amplification of at least part of said target sequences comprise or consist of at least 15 consecutive nucleotides of the sequences as defined in SEQ ID NOS: 40-43.

In a preferred embodiment, at least the presence of liver worms, such as Clonorchis sinensis, Opisthorchis spp., and Metorchis spp. are detected in the method, wherein the target sequence is at least as defined by SEQ ID NO:16. More preferably, a primer pair set allowing amplification of at least part of said target sequences comprise or consist of at least 15 consecutive nucleotides of the sequences as defined in SEQ ID NOS: 44 and 45.

In a preferred embodiment, the presence of at least Hymenolepis nana, Hymenolepis diminuta and liver worms Clonorchis sinensis, Opisthorchis spp., and Metorchis spp are detected in the method, wherein the target sequences are at least as defined by SEQ ID NOS:1, 2 and 16. More preferably, a primer pair set allowing amplification of at least part of said target sequences comprise or consist of at least 15 consecutive nucleotides of the sequences as defined in SEQ ID NOS:17, 18, 19, 44 and 45.

In another preferred embodiment, the presence of at least Enterocytozoon bieneusi, Enterobius vermicularis, and Schistosoma mansoni are detected in the method, wherein the target sequences are at least as defined by SEQ ID NOS:7, 8 and 13. More preferably, a primer pair set allowing amplification of at least part of said target sequences comprise or consist of at least 15 consecutive nucleotides of the sequences as defined in SEQ ID NOS:28, 29, 30, 31, 38 and 39.

In a preferred embodiment, at least the presence of Ancylostoma duodenalis is detected in the method, wherein the target sequences are at least as defined by SEQ ID NOS:46 and 47. More preferably, a primer pair set allowing amplification of at least part of said target sequences comprise or consist of at least 15 consecutive nucleotides of the sequences as defined in SEQ ID NOS: 48-51.

The present invention is further directed to the use of nucleotide primers, primer pairs or probes as defined above for determining the presence of intestinal parasites in a sample.

The present invention also provides kits for the detection of the presence of intestinal parasites in a sample. Such a kit comprises primer pairs selected from the group consisting of primer pairs as defined above. The kit may further comprise a probe selected from the probes as defined above. The use of the primer pairs and probes are described above and in the Example below. Preferably, said kit comprises means for a real-time polymerase chain reaction, such as labelled probes, polymerase enzymes, buffers and nucleotides.

Unless explained otherwise, all technical and scientific terms used herein have the same meaning as commonly understood to one of ordinary skill in the art to which this disclosure belongs. Although methods and materials similar or equivalent to those described herein can be used in the practice or testing of the present disclosure, suitable methods and materials are described below. The materials, methods, and examples are illustrative only and not intended to be limiting.

### Example 1

This example describes results from a proof-of-concept study of the detection of Hymenolepis nana, Hymenolepis diminuta, Fasciolopsis buski, Encephalitozoon spp. (such as E. intestinalis, E. cuniculi and E. hellem), Enterocytozoon bieneusi, Enterobius vermicularis, Diphyllobothrium latum, Diphyllobothrium nihonkaiense, Schistosoma mansoni, Blastocystis hominis and liver worms, such as Clonorchis sinensis, Opisthorchis spp., and Metorchis spp. in a proprietary multiplex qPCR assay. Sample material for this designed assay is a spiked stool sample.

Materials and Methods

qPCR Reagents:

Mobidiag's qPCR Mastermix (MM)

Assay mixture consisting of parasite target specific primers as defined in Table 2 and probes as defined above.

Devices:

BIO-RAD CFX96

PCR Setup

In reaction:

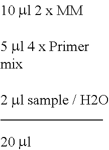

PCR Protocol:

| | | | |
|---|---|---|---|
| 95° C. | 10 | min | 45x |
| 95° C. | 15 | s | |
| 63° C. | 1 | min | |

Two-step qPCR with detection by labelled probes.

Samples:

Spiked samples, representing the pathogens listed above, in a stool background. Samples have been collected from commercially available biobanks (such as ATCC) or from Mobidiag sample storage facilities and the analyses are performed in a series of ten-fold sample dilutions.

Results

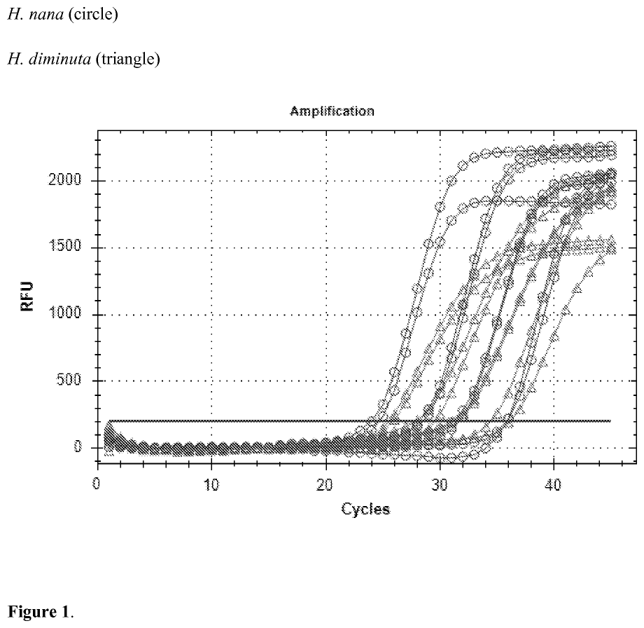

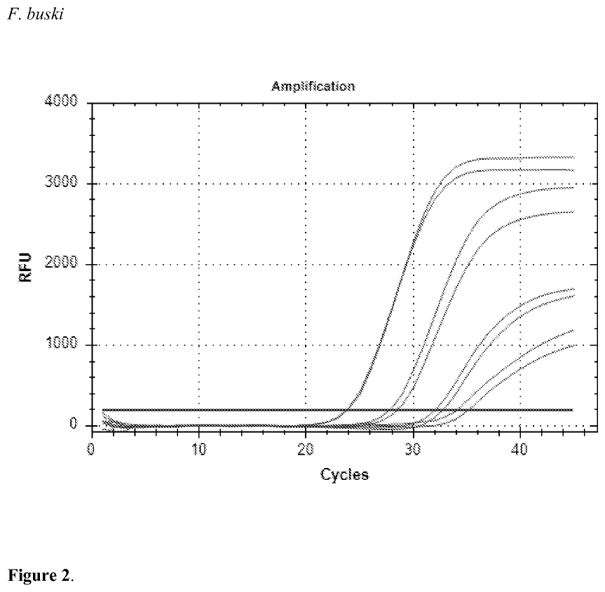

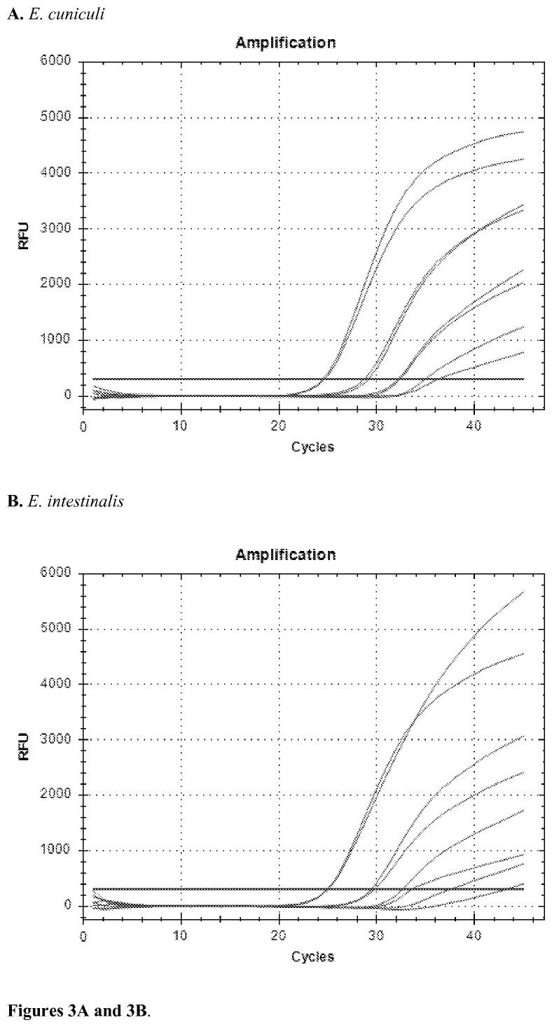

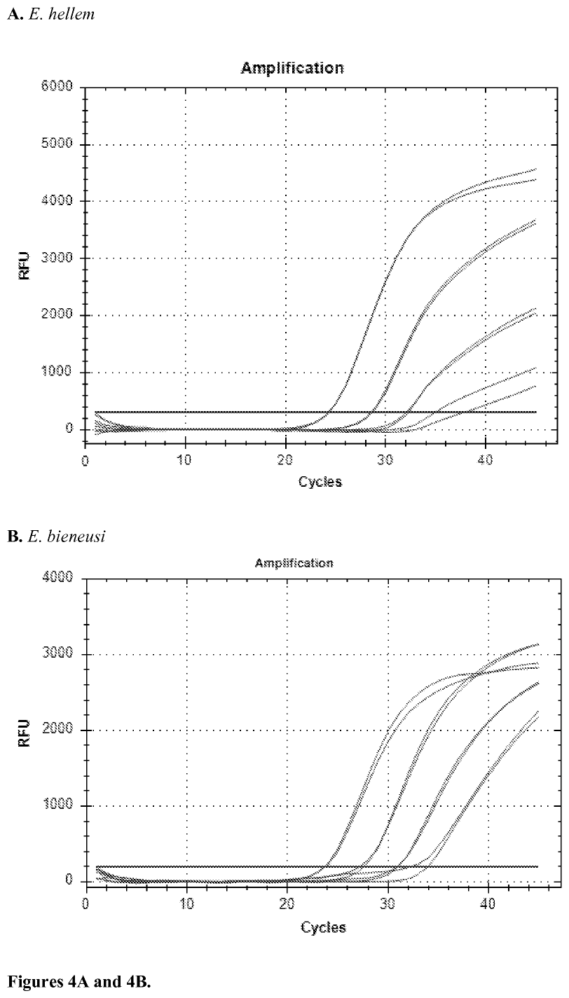

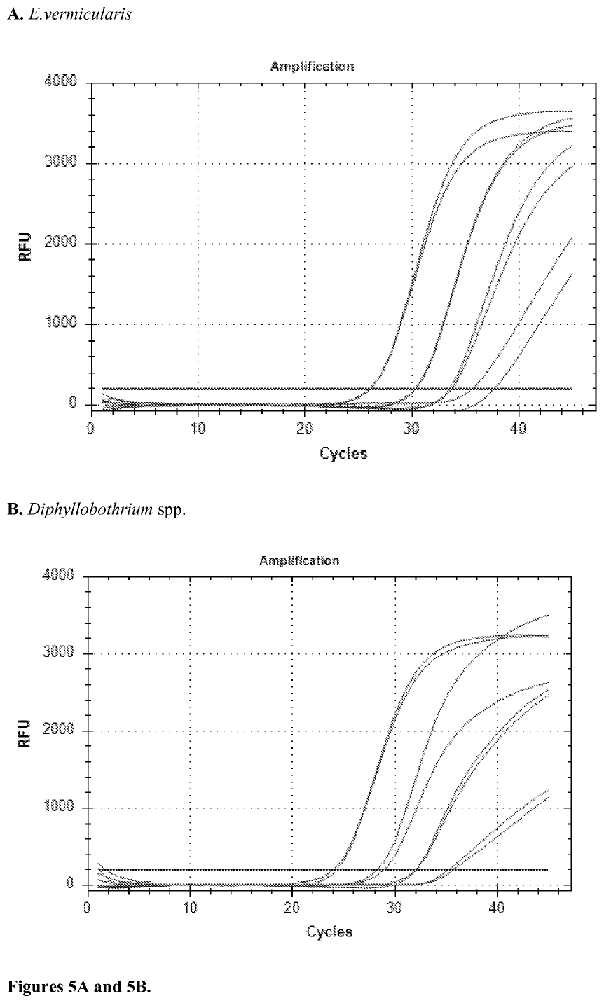

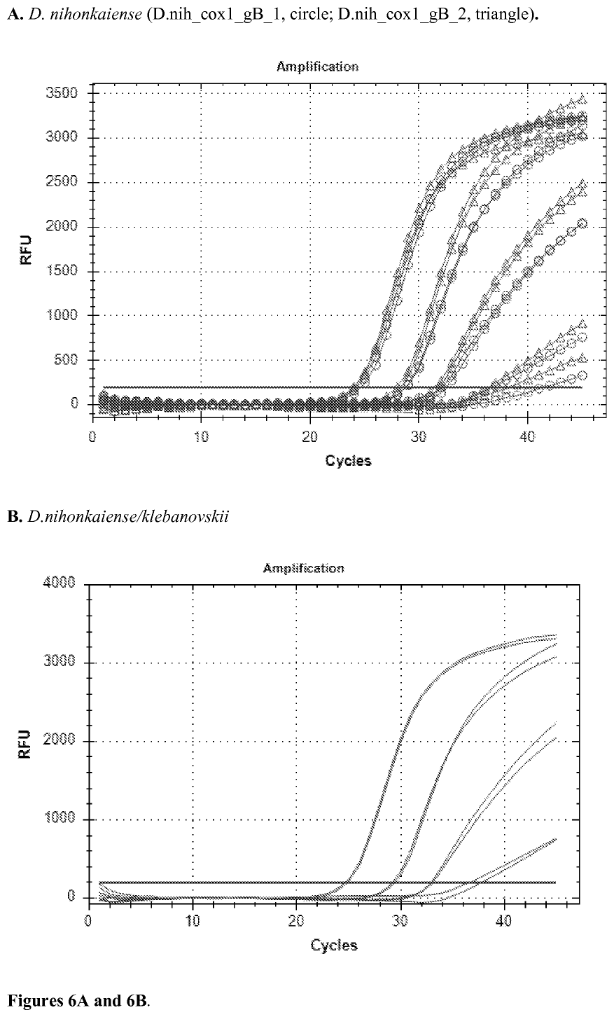

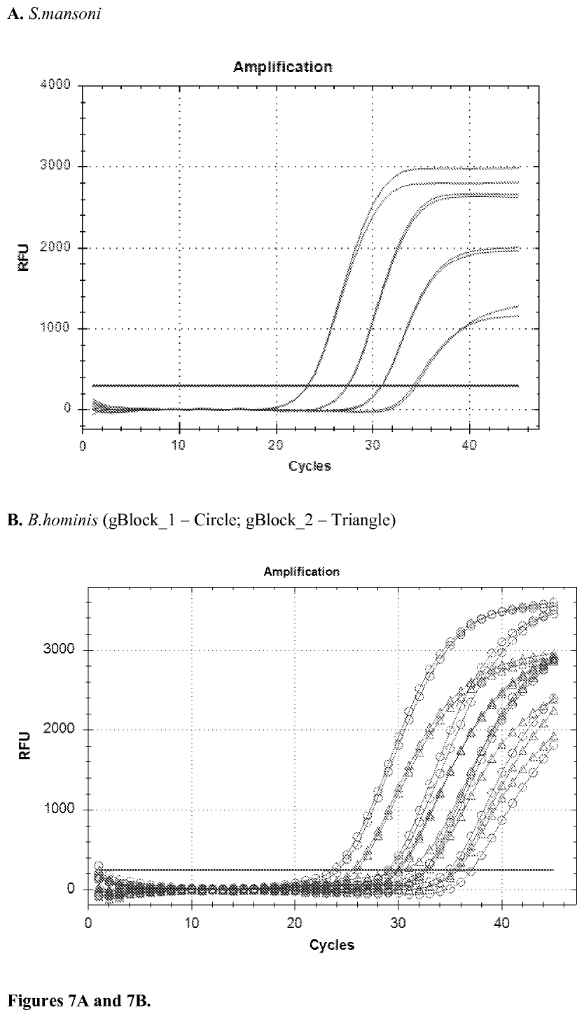

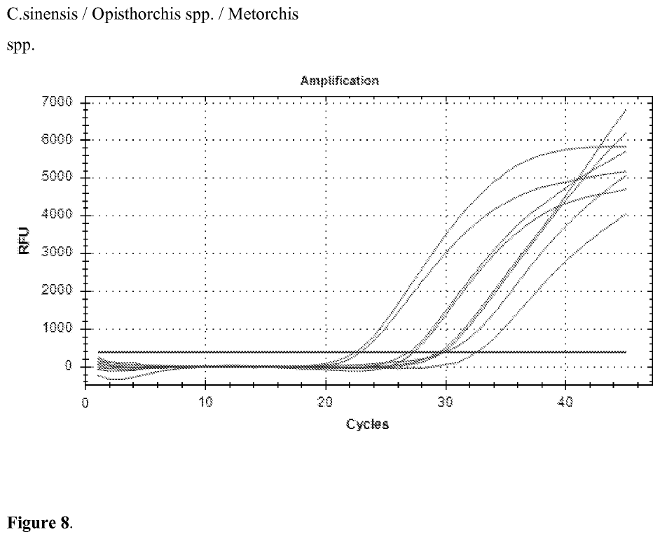

All targets were detected in all sample concentrations in a high multiplexing condition (FIGS. 1-8).

### Example 2

This example describes results from a study of potential false positive results in the intestinal parasites qPCR assay due to a cross-reaction. Sample material for this designed assay is preferably stool sample. Therefore, pathogens other than parasites (bacteria and viruses) associated with gastrointestinal infections, and which are not covered by assay panel, can cause potential cross-reaction. Also, other eukaryotic microbes may cross-react.

Materials and Methods

qPCR Reagents:

Mobidiag's qPCR Mastermix (MM)

Assay mixture consisting of parasite target specific primers as defined in Table 2 and probes as defined above.

Devices:

BIO-RAD CFX96

PCR Setup

In reaction:

10 μl2×MM

5 μl 4×Primer mix

2 μl sample/H2O

20 μl

PCR Protocol

A two-step qPCR with detection by labelled probes:

| | | | |
|---|---|---|---|
| 95° C. | 10 | min | 45x |
| 95° C. | 15 | s | |
| 63° C. | 1 | min | |

Samples:

In total, 61 living or attenuated microbes or extracted DNA samples from different micro-organisms (Table 3). Strains have been mainly collected from commercially available biobanks (ATCC, DSMZ, Microbiologics Qnostics and Vircell). All samples are analysed at high (>10⁸ CFU/mL) concentrations.

TABLE 3
Cross-reaction results.

| Target | Result |
|---|---|
| Acanthamoeba castellanii | Negative |
| Neospora caninum | Negative |
| Perkinsus marinus | Negative |
| Prototheca wickerhamii | Negative |
| Tetrahymena thermophila | Negative |
| Aspergillus fumigatus | Negative |
| Debaryomyces hansenii | Negative |
| Eremothecium gossypii | Negative |
| Malassezia globosa | Negative |
| Malassezia pachydermatis | Negative |
| Penicillium rubens | Negative |
| Saprolegnia diclina | Negative |
| Trichoderma reesei | Negative |
| Trichophyton interdigitale | Negative |
| Geotrichum candidum | Negative |
| Iodamoeba butschlii | Negative |
| Endolimax nana | Negative |
| Entamoeba coli | Negative |
| Entamoeba dispar | Negative |
| Plasmodium falciparum | Negative |
| Plasmodium malariae | Negative |
| Aspergillus fumigatus | Negative |
| Candida albicans | Negative |
| Candida glabrata | Negative |
| Candida krusei | Negative |
| Fusarium solani | Negative |
| Saccharomyces cervisiae | Negative |
| Aeromonas hydrophila | Negative |
| Escherichia coli, non toxigenic | Negative |
| Escherichia coli, EAEC | Negative |
| Escherichia coli, EIEC | Negative |
| Escherichia coli, EPEC | Negative |
| Escherichia coli, ETEC | Negative |
| Bacillus cereus | Negative |
| Bacteroides fragilis | Negative |
| Campylobacter coli | Negative |
| Campylobacter jejuni | Negative |
| Clostridium difficile | Negative |
| Clostridium perfringens | Negative |
| Clostridium sordellii | Negative |
| Enterobacter cloacae | Negative |
| Enterococcus faecalis | Negative |
| Enterococcus faecium | Negative |
| Helicobacter pylori | Negative |
| Klebsiella pneumoniae | Negative |
| Lactobacillus acidophilus | Negative |
| Proteus vulgaris | Negative |
| Pseudomonas aeruginosa | Negative |
| Salmonella enterica subsp. enterica, Typhimurium | Negative |
| Shigella sonnei | Negative |
| Serratia marcescens | Negative |
| Staphylococcus aureus | Negative |
| Staphylococcus epidermidis | Negative |
| Streptococcus bovis | Negative |
| Vibrio parhaemolyticus | Negative |
| Yersinia enterocolitica subsp. enterocolitica | Negative |
| Yersinia pseudotuberculosis | Negative |
| Cytomegalovirus | Negative |
| Human herpesvirus 1 | Negative |
| Human herpesvirus 2 | Negative |
| Human adenovirus 41 | Negative |

Results

The cross-reactivity test showed no false positives (see Table 3 above).

### Example 3

For the experiment, two set of samples were used: a set of (n=8) known Encephalitozoon spp. positive samples from clinical origin (one per patient) and a set of (n=104) stool samples negative for Encephalitozoon spp. (see Table 4). Positive samples were prepared by spiking the known strains into negative stool background in clinically relevant concentrations. In total, 120 Novodiag cartridges (Mobidiag, Finland) were run.

Sample Quantitation

The known Encephalitozoon samples from commercially available biobank (ATCC) were quantified in CFX96 qPCR instrument against known standard DNA sample of the same target diluted in a 10-fold fashion. The standard series ranged from 200 to 200 000 c/μL. The final “clinical” samples were prepared by spiking the primary Encephalitozoon spp. samples into eSwab-stool-suspension in clinically relevant concentration (ranging from 100 to 80 000 cells/mL).

Sample Analysis

Each sample (positive and negative) were pre-treated and run in the Novodiag instrument.

TABLE 4

| Samples | ATTC Number | Comment |
|---|---|---|
| E. intestinalis {sample 1) | ATCC 50651 | From clinical origin |
| E. intestinalis (sample 2) | ATCC 50507 | From clinical origin |
| E. intestinalis (sample 3) | ATCC 50506 | From clinical origin |
| E. cuniculi (sample 4) | ATCC 50503 | From clinical origin |
| E. cuniculi (sample 5) | ATCC 50789 | From clinical origin |
| E. cuniculi (sample 6) | ATCC 50612 | From clinical origin |
| E. hellem (sample 7) | ATCC 50504 | From clinical origin |
| E. hellem (sample 8) | ATCC 80451 | From clinical origin |
| Samples (n = 104) negative | N/A | Clinical pseudonymised |
| for Encephalitozoon spp. | | left-over samples and |
| | | MDE biobank samples |

Oligonucleotides

Encephalitozoon Assay mix comprised the following oligonucleotides:

Encephalitozoon_sp_18S_F3.1 (SEQ ID NO:22)

Encephalitozoon_sp_18S_F3.2 (SEQ ID NO:24)

Encephalitozoon_sp_18S_F3.3 (SEQ ID NO:26)

Encephalitozoon_sp_18S_P2.1 as

Encephalitozoon_sp_18S_P2.2 as

Encephalitozoon_sp_18S_R2.1 (SEQ ID NO:23)

Encephalitozoon_sp_18S_R2.2 (SEQ ID NO:25)

Encephalitozoon_sp_18S_R2.3 (SEQ ID NO:27)

Results

The results of positive samples with cell count approximation are presented in Table 5 below:

| | | | Copies/mL | | | |
|---|---|---|---|---|---|---|
| | | | converted | | Clinically relevant | |
| | Detected | | into cells/ml | | concentrations | |
| | (conc. yielding | | (conversion | | commonly | |
| Encephalitozoon samples: | positive call) | | factor 11)¹ | | found in stools^{2, 3} | |
| E. intestinalis ATCC 50651 | 32000 | c/mL | ~3000 | cells/mL | 230-78000 | cells/mL |
| E. intestinalis ATCC 50507 | 6400 | c/mL | ~600 | cells/mL | 231-78000 | cells/mL |
| E. cuniculi ATCC 50503 | 1800 | c/mL | ~200 | cells/mL | 100-1000 | cells/mL |
| E. hellem ATCC 50504 | 1900 | c/mL | ~200 | cells/mL | 180-3600 | cells/mL |
| E. hellem ATCC 50451 | 32000 | c/mL | ~3000 | cells/mL | 180-3600 | cells/mL |
| E. intestinalis ATCC 50506 | 3500 | c/mL | ~300 | cells/mL | 231-78000 | cells/mL |
| E. cuniculi ATCC 50789 | 3500 | c/mL | ~300 | cells/mL | 100-1000 | cells/mL |
| E. cuniculi ATCC 50612 | 1800 | c/mL | ~200 | cells/mL | 100-1000 | cells/mL |

¹Conversion factor 11 comes from the number of copies of 18S genes found in Encephalitozoon spp. nuclei. Biderre C, Peyretaillade E, Duffieux F, Peyret P, Méténier G, Vivarès C. The rDNA Unit of Encephalitozoon cuniculi (Microsporidia): Complete 23S Sequence and Copy Number. J Eukaryot Microbiol. Nov-Dec 1997; 44(6): 76S.
² Graczyk TK, Johansson MA, Tamang L, Visvesvara GS, Moura LS, DaSilva AJ, Girouard AS, Matos O. Retrospective Species Identification of Microsporidian Spores in Diarrheic Fecal Samples from Human Immunodeficiency Virus/AIDS Patients by Multiplexed Fluorescence In Situ Hybridization. J Clin Microbiol. 2007 Apr; 45(4): 1255-60.
³ Kahler AM, Thurston-Enriquez JA. Human pathogenic microsporidia detection in agricultural samples: method development and assessment. Parasitol Res. 2007 Feb; 100(3): 529-38.

The final results are summarized below in Table 6:

| | Novodiag NVD Stool Parasites results | | | | | | | | |
|---|---|---|---|---|---|---|---|---|---|
| | | | | | Sens | Spec | PPV | NPV | Accuracy |
| Targets | TP | FP | FN | TN | (95% CI) | (95% CI) | (95% CI) | (95% CI) | (95% CI) |
| Encephalitozoon spp. | 8 | 0 | 0 | 104 | 100% | 100% | 100% | 100% | 100% |
| | | | | | (63.1-100%) | (96.5-100%) | (63.1-100%) | (96.5-100%) | (96.8-100%) |
| E. cuniculi | 3 | 0 | 0 | 104 | 100% | 100% | 100% | 100% | 100% |
| | | | | | (29.2-100%) | (96.5-100%) | (29.2-100%) | (96.5-100%) | (96.6-100%) |
| E. hellem | 2 | 0 | 0 | 104 | 100% | 100% | 100% | 100% | 100% |
| | | | | | (15.8-100%) | (96.5-100%) | (15.8-100%) | (96.5-100%) | (96.6-100%) |
| E. intestinalis | 3 | 0 | 0 | 104 | 100% | 100% | 100% | 100% | 100% |
| | | | | | (29.2-100%) | (96.5-100%) | (29.2-100%) | (96.5-100%) | (96.6-100%) |

Overall sensitivity and specificity of the assay for detection of Encephalitozoon spp. with spiked and Encephalitozoon spp. negative stool samples was 100% (95% CI 63.1-100%) and 100% (95% CI 96.5-100%), respectively.

Overall positive predictive value (PPV) and negative predictive value (NPV) was 100% (95% CI 63.1-100%) and 100% (95% CI 96.5-100%), respectively.

### Example 4

This experiment was conducted with Ancylostoma duodenale primers as described in SEQ ID NOS:48-51 and the results were compared to the reference O&P microscopic method.

The final results are:

| | Novodiag NVD Stool Parasites results vs. O&P microscopy | | | | | | | | | | |
|---|---|---|---|---|---|---|---|---|---|---|---|
| | | | | | Sens | Spec | PPV | NPV | Accuracy | | |
| Target | TP | FP | FN | TN | (95% CI) | (95% CI) | (95% CI) | (95% CI) | (95% CI) | PLR | NLR |
| Ancylostoma | 3 | 0 | 0 | 93 | 100% | 100% | 100% | 100% | 100% | N/A | 0 |
| duodenale | | | | | (29.2-100%) | (96.1-100%) | (29.2-100%) | (96.1-100%) | (96.2-100%) | | |

PLR = Positive Likelihood Ratio. Since no clinical data were obtained for this study, the likelihood ratio and correctness are estimations only.
NLR = Negative Likelihood Ratio. Since no clinical data were obtained for this study, the likelihood ratio and correctness are estimations only.
N/A = cannot be calculated since sens. and spec. are 100%.

Overall sensitivity and specificity of the NVD SP assay for detection of Ancylostoma duodenale from unpreserved stool samples was 100% (95% CI 29.2-100%) and 100% (95% CT 96.1-100%), respectively.

Overall PPV and NPV was 100% (95% CI 29.2-100%) and 100% (95% CI 96.1-100%), respectively.

One invalid run was observed from the sample set (1/96) yielding 1% invalidity rate.

### REFERENCES

Garcia, Lynne S., Michael Arrowood, Evelyne Kokoskin, Graeme P. Paltridge, Dylan R. Pillai, Gary W. Procop, Norbert Ryan, Robyn Y. Shimizu, and Govinda Visvesvarab, Laboratory Diagnosis of Parasites from the Gastrointestinal Tract, Clinical Microbiology Reviews, January 2018, Volume 31, Issue 1, e00025-17.

1. A method for detecting the presence or absence of one or more intestinal parasites in a biological sample comprising the steps of:
i) contacting the sample or nucleic acid isolated therefrom with a set of oligonucleotide primers comprising one or more primer pairs that binds to a target sequence selected from SEQ ID NOs: 1-16 and 46-47 and allow amplification of at least part of the target sequence in step ii);
ii) performing a nucleic acid amplification reaction to specifically amplify the target sequences of the intestinal parasite(s) in the sample; and
iii) detecting the presence of an amplified target sequence, wherein the presence of the target sequence is indicative of the presence of one or more intestinal parasites in the sample,
wherein the one or more intestinal parasites is selected from Hymenolepis nana, Hymenolepis diminuta, Fasciolopsis buski, Encephalitozoon spp., Enterocytozoon bieneusi, Enterobius vermicularis, Diphyllobothrium latum, Diphyllobothrium nihonkaiense, Schistosoma mansoni, Blastocystis hominis, Ancylostoma duodenale and liver worms.

2. The method according to claim 1, wherein the set of oligonucleotide primers comprises one or more primer pairs selected from of:
a) a primer pair for detecting Hymenolepis nana and Hymenolepis diminuta comprising a forward primer comprising or consisting of at least 15 consecutive nucleotides of AATTCCTGATGCTTTTGGGTTTTATG (SEQ ID NO:17) and a reverse primer comprising or consisting of at least 15 consecutive nucleotides of AGAACACTGCCGTCTTTACATCTAA (SEQ ID NO:18);
b) a primer pair for detecting Hymenolepis nana and Hymenolepis diminuta comprising a forward primer comprising or consisting of at least 15 consecutive nucleotides of AATTCCTGATGCTTTTGGGTTTTATG (SEQ ID NO:17) and a reverse primer comprising or consisting of at least 15 consecutive nucleotides of AAATACAGCCGTCTTAACATCCAA (SEQ ID NO:19);
c) a primer pair for detecting Fasciolopsis buski comprising a forward primer comprising or consisting of at least 15 consecutive nucleotides of CACTGTTCAAGTGGTATTGATTG (SEQ ID NO:20) and a reverse primer comprising or consisting of at least 15 consecutive nucleotides of CCAGGTTATCAGTCCTACCC (SEQ ID NO:21);
d) a primer pair for detecting E. intestinalis, E. cuniculi, and E. hellem comprising a forward primer comprising or consisting of at least 15 consecutive nucleotides of CTGAGTCCTGAGTGTTAGATAAGA (SEQ ID NO:22) and a reverse primer comprising or consisting of at least 15 consecutive nucleotides of CTAATGCCAATCAATCCCGTG (SEQ ID NO:23);
e) a primer pair for detecting E. intestinalis, E. cuniculi, and E. hellem comprising a forward primer comprising or consisting of at least 15 consecutive nucleotides of GTCCTTCGTGTTAGATAAGATATAAGTC (SEQ ID NO:24) and a reverse primer comprising or consisting of at least 15 consecutive nucleotides of AGATAATGCCAATCAATCCCATG (SEQ ID NO:25);
f) a primer pair for detecting E. intestinalis, E. cuniculi, and E. hellem comprising a forward primer comprising or consisting of at least 15 consecutive nucleotides of GACGAAGATTGAGAGGTCTGA (SEQ ID NO:26) and a reverse primer comprising or consisting of at least 15 consecutive nucleotides of CTAATGCCTATCAATCCCGTG (SEQ ID NO:27);
g) a primer pair for detecting Enterocytozoon bieneusi comprising a forward primer comprising or consisting of at least 15 consecutive nucleotides of GAGTGTAGTATAGACTGGCGAA (SEQ ID NO:28) and a reverse primer comprising or consisting of at least 15 consecutive nucleotides of TCGTCCTTGATCCTAAGATACG (SEQ ID NO:29);
h) a primer pair for detecting Enterobius vermicularis comprising a forward primer comprising or consisting of at least 15 consecutive nucleotides of GCAGAGCTTTTCCAAAATTTATTTCC (SEQ ID NO:30) and a reverse primer comprising or consisting of at least 15 consecutive nucleotides of CCCAAGTTTGAGGTAATTTCTCG (SEQ ID NO:31);
i) a primer pair for detecting Diphyllobothrium latum and Diphyllobothrium nihonkaiense comprising a forward primer comprising or consisting of at least 15 consecutive nucleotides of CCAGTTATTACTGGTGTAAGATTGAA (SEQ ID NO:32) and a reverse primer comprising or consisting of at least 15 consecutive nucleotides of TCAAGCATAACCTGACTCATATAC (SEQ ID NO:33);
j) a primer pair for detecting Diphyllobothrium latum and Diphyllobothrium nihonkaiense comprising a forward primer comprising or consisting of at least 15 consecutive nucleotides of CCAGTTATTACTGGTGTAAGATTGAA (SEQ ID NO:34) and a reverse primer comprising or consisting of at least 15 consecutive nucleotides of TCAAGCATAACCTGACTCATATAC (SEQ ID NO:35);
k) a primer pair for detecting Diphyllobothrium latum and Diphyllobothrium nihonkaiense comprising a forward primer comprising or consisting of at least 15 consecutive nucleotides of CCAGTTATTACAGGTGTGAGATTG (SEQ ID NO:36) and a reverse primer comprising or consisting of at least 15 consecutive nucleotides of CAAGCATAACCCGACTCGTA (SEQ ID NO:37);
l) a primer pair for detecting Diphyllobothrium latum and Diphyllobothrium nihonkaiense comprising a forward primer comprising or consisting of at least 15 consecutive nucleotides of CCAGTTATTACAGGTGTGAGATTG (SEQ ID NO:36) and a reverse primer comprising or consisting of at least 15 consecutive nucleotides of CAAGCATAACCCGACTCGTA (SEQ ID NO:37);
m) a primer pair for detecting Schistosoma mansoni comprising a forward primer comprising or consisting of at least 15 consecutive nucleotides of AGGTGTTTTCATGACTTTATATGTTGA (SEQ ID NO:38) and a reverse primer comprising or consisting of at least 15 consecutive nucleotides of AGCAGATGCAGATAAAGCCA (SEQ ID NO:39);
n) a primer pair for detecting Blastocystis hominis comprising a forward primer comprising or consisting of at least 15 consecutive nucleotides of CAGCTTTCGATGGTAGTGTATTG (SEQ ID NO:40) and a reverse primer comprising or consisting of at least 15 consecutive nucleotides of GGCTCCCTCTCCGAAATC (SEQ ID NO:41);
o) a primer pair for detecting Blastocystis hominis comprising a forward primer comprising or consisting of at least 15 consecutive nucleotides of TCAGCTTTCGATGGTAGTATATGG (SEQ ID NO:42) and a reverse primer comprising or consisting of at least 15 consecutive nucleotides of GGCTCCCTCTCCGAAATC (SEQ ID NO:43);
p) a primer pair for detecting liver worms comprising a forward primer comprising or consisting of at least 15 consecutive nucleotides of AGCTCGTAGTTGGATCTGG (SEQ ID NO:44) and a reverse primer comprising or consisting of at least 15 consecutive nucleotides of CCACCAATCATGCTAACACC (SEQ ID NO:45);
q) a primer pair for detecting Ancylostoma duodenale comprising a forward primer comprising or consisting of at least 15 consecutive nucleotides of CAGTGTAGCTTGTGGCAC (SEQ ID NO:48) and a reverse primer comprising or consisting of at least 15 consecutive nucleotides of CAGCTAACGTACATGTTGCAATA (SEQ ID NO:49); and
r) a primer pair for detecting Ancylostoma duodenale comprising a forward primer comprising or consisting of at least 15 consecutive nucleotides of ACAGTGCAGCTTGTGGCA (SEQ ID NO:50) and a reverse primer comprising or consisting of at least 15 consecutive nucleotides of CAGCCAACGTACATGTTGCAATA (SEQ ID NO:51).

3. The method according to claim 2, wherein the set of oligonucleotides comprises primer pairs (a)-(p).

4. The method according to claim 3, wherein the forward and reverse primers of primer pairs (a)-(p) consist of at least 15 contiguous nucleotides of the nucleotide sequences of SEQ ID NOS: 17-45, respectively.

5. (canceled)

6. The method according to claim 1, wherein said biological sample is a stool sample or a food sample.

7. (canceled)

8. The method according to claim 2, wherein wherein the method comprises contacting the product of the nucleic acid amplification reaction with one or more probes selected from:
a) a probe for detecting Hymenolepis nana and Hymenolepis diminuta comprising or consisting of a sequence that is identical or complementary to at least 10 consecutive nucleotides of 5′-AGTGTGCTTAGGTTGTAGTGTGTGGGCTCATC-3′ (SEQ ID NO:52);
b) a probe for detecting Hymenolepis nana and Hymenolepis diminuta comprising or consisting of a sequence that is identical or complementary to at least 10 consecutive nucleotides of 5′-TGTTTGCCATGTTTTCTATTGTTTGTTTAGG-3′ (SEQ ID NO:53);
c) a probe for detecting Fasciolopsis buski comprising or consisting of a sequence that is identical or complementary to at least 10 consecutive nucleotides of 5′-TTCGCCCATTCTTTGCCATTGCCC-3′ (SEQ ID NO:54);
d) a probe for detecting E. intestinalis, E. cuniculi, and E. hellem comprising or consisting of a sequence that is identical or complementary to at least 10 consecutive nucleotides of 5′-CTGATCCTGCTGCTGGTTCTCCAACAG-3′ (SEQ ID NO:55);
e) a probe for detecting E. intestinalis, E. cuniculi, and E. hellem comprising or consisting of a sequence that is identical or complementary to at least 10 consecutive nucleotides of 5′-ATGATCCTGCTAATGGTTCTCCAACAGCA-3′ (SEQ ID NO:56);
f) a probe for detecting E. intestinalis, E. cuniculi, and E. hellem comprising or consisting of a sequence that is identical or complementary to at least 10 consecutive nucleotides of 5′-ATGATCCTGCTAATGGTTCTCCAACAGCA-3′ (SEQ ID NO:57);
g) a probe for detecting Enterocytozoon bieneusi comprising or consisting of a sequence that is identical or complementary to at least 10 consecutive nucleotides of 5′-AGTGTCGCCTTCGCCTCCGTTAG-3′ (SEQ ID NO:58);
h) a probe for detecting Enterobius vermicularis comprising or consisting of a sequence that is identical or complementary to at least 10 consecutive nucleotides of 5′-TCCGGCTCAGACATGAACATCAGTGAGTCT-3′ (SEQ ID NO:59);
i) a probe for detecting Diphyllobothrium latum and Diphyllobothrium nihonkaiense comprising or consisting of a sequence that is identical or complementary to at least 10 consecutive nucleotides of 5′-ACACGACGTGGTAAACCGCACACA-3′ (SEQ ID NO:60);
j) a probe for detecting Diphyllobothrium latum and Diphyllobothrium nihonkaiense comprising or consisting of a sequence that is identical or complementary to at least 10 consecutive nucleotides of 5′-ACACGACGTGGTAAACCGCACACA-3′ (SEQ ID NO:61);
k) a probe for detecting Diphyllobothrium latum and Diphyllobothrium nihonkaiense comprising or consisting of a sequence that is identical or complementary to at least 10 consecutive nucleotides of 5′-ACACGACGTGGTAAACCGCACACA-3′ (SEQ ID NO:62);
l) a probe for detecting Diphyllobothrium latum and Diphyllobothrium nihonkaiense comprising or consisting of a sequence that is identical or complementary to at least 10 consecutive nucleotides of 5′-ACACGACGTGGTAAACCGCACACA-3′ (SEQ ID NO:63);
m) a probe for detecting Schistosoma mansoni comprising or consisting of a sequence that is identical or complementary to at least 10 consecutive nucleotides of 5′-CCCCTGTGACACCACCAACCGT-3′ (SEQ ID NO:64);
n) a probe for detecting Blastocystis hominis comprising or consisting of a sequence that is identical or complementary to at least 10 consecutive nucleotides of 5′-AAATTCTTCGTTACCCGTTACTGCCATGGT-3′ (SEQ ID NO:65);
o) a probe for detecting Blastocystis hominis comprising or consisting of a sequence that is identical or complementary to at least 10 consecutive nucleotides of 5′-AAATTCTTCGTTACCCGTTACTGCCATGGT-3′ (SEQ ID NO:66);
p) a probe for detecting liver worms comprising or consisting of a sequence that is identical or complementary to at least 10 consecutive nucleotides of 5′-TTGCTCGTATTCCTGGCCTGGTTCA-3′ (SEQ ID NO:67).

9. (canceled)

10. The method according to claim 1, wherein the method comprises detecting the presence or absence of at least Hymenolepis nana, Hymenolepis diminuta and liver worms, and wherein the target sequences comprise at least SEQ ID Nos: 1, 2 and 16.

11. The method according to claim 10, wherein the set of oligonucleotide primers comprises:
a) a primer pair for detecting Hymenolepis nana and Hymenolepis diminuta comprising a forward primer comprising or consisting of at least 15 consecutive nucleotides of AATTCCTGATGCTTTTGGGTTTTATG (SEQ ID NO:17) and a reverse primer comprising or consisting of at least 15 consecutive nucleotides of AGAACACTGCCGTCTTTACATCTAA (SEQ ID NO:18);
a primer pair for detecting Hymenolepis nana and Hymenolepis diminuta comprising a forward primer comprising or consisting of at least 15 consecutive nucleotides of AATTCCTGATGCTTTTGGGTTTTATG (SEQ ID NO:17) and a reverse primer comprising or consisting of at least 15 consecutive nucleotides of AAATACAGCCGTCTTAACATCCAA (SEQ ID NO:19); and
c) a primer pair for detecting liver worms comprising a forward primer comprising or consisting of at least 15 consecutive nucleotides of AGCTCGTAGTTGGATCTGG (SEQ ID NO:44) and a reverse primer comprising or consisting of at least 15 consecutive nucleotides of CCACCAATCATGCTAACACC (SEQ ID NO:45).

12. The method according to claim 1, wherein the method comprises detecting the presence or absence at least Enterocytozoon bieneusi, Enterobius vermicularis, and Schistosoma mansoni, and wherein the target sequences comprise SEQ ID Nos: 7, 8 and 13.

13. The method according to claim 12, wherein the set of oligonucleotide primers comprises:
a) a primer pair for detecting Enterocytozoon bieneusi comprising a forward primer comprising or consisting of at least 15 consecutive nucleotides of GAGTGTAGTATAGACTGGCGAA (SEQ ID NO:28) and a reverse primer comprising or consisting of at least 15 consecutive nucleotides of TCGTCCTTGATCCTAAGATACG (SEQ ID NO:29);
b) a primer pair for detecting Enterobius vermicularis comprising a forward primer comprising or consisting of at least 15 consecutive nucleotides of GCAGAGCTTTTCCAAAATTTATTTCC (SEQ ID NO:30) and a reverse primer comprising or consisting of at least 15 consecutive nucleotides of CCCAAGTTTGAGGTAATTTCTCG (SEQ ID NO:31); and
c) a primer pair for detecting Schistosoma mansoni comprising a forward primer comprising or consisting of at least 15 consecutive nucleotides of AGGTGTTTTCATGACTTTATATGTTGA (SEQ ID NO:38) and a reverse primer comprising or consisting of at least 15 consecutive nucleotides of AGCAGATGCAGATAAAGCCA (SEQ ID NO:39).

14. (canceled)

15. (canceled)

16. (canceled)

17. A composition comprising a set of oligonucleotide primers and probes, wherein the probes each comprise a detectable label, and wherein the set of primers comprises one or more primer pairs selected from:
a) a primer pair for detecting Hymenolepis nana and Hymenolepis diminuta comprising a forward primer comprising or consisting of at least 15 consecutive nucleotides of AATTCCTGATGCTTTTGGGTTTTATG (SEQ ID NO:17) and a reverse primer comprising or consisting of at least 15 consecutive nucleotides of AGAACACTGCCGTCTTTACATCTAA (SEQ ID NO:18);
b) a primer pair for detecting Hymenolepis nana and Hymenolepis diminuta comprising a forward primer comprising or consisting of at least 15 consecutive nucleotides of AATTCCTGATGCTTTTGGGTTTTATG (SEQ ID NO:17) and a reverse primer comprising or consisting of at least 15 consecutive nucleotides of AAATACAGCCGTCTTAACATCCAA (SEQ ID NO:19);
c) a primer pair for detecting Fasciolopsis buski comprising a forward primer comprising or consisting of at least 15 consecutive nucleotides of CACTGTTCAAGTGGTATTGATTG (SEQ ID NO:20) and a reverse primer comprising or consisting of at least 15 consecutive nucleotides of CCAGGTTATCAGTCCTACCC (SEQ ID NO:21);
d) a primer pair for detecting E. intestinalis, E. cuniculi, and E. hellem comprising a forward primer comprising or consisting of at least 15 consecutive nucleotides of CTGAGTCCTGAGTGTTAGATAAGA (SEQ ID NO:22) and a reverse primer comprising or consisting of at least 15 consecutive nucleotides of CTAATGCCAATCAATCCCGTG (SEQ ID NO:23);
e) a primer pair for detecting E. intestinalis, E. cuniculi, and E. hellem comprising a forward primer comprising or consisting of at least 15 consecutive nucleotides of GTCCTTCGTGTTAGATAAGATATAAGTC (SEQ ID NO:24) and a reverse primer comprising or consisting of at least 15 consecutive nucleotides of AGATAATGCCAATCAATCCCATG (SEQ ID NO:25);
f) a primer pair for detecting E. intestinalis, E. cuniculi, and E. hellem comprising a forward primer comprising or consisting of at least 15 consecutive nucleotides of GACGAAGATTGAGAGGTCTGA (SEQ ID NO:26) and a reverse primer comprising or consisting of at least 15 consecutive nucleotides of CTAATGCCTATCAATCCCGTG (SEQ ID NO:27);
g) a primer pair for detecting Enterocytozoon bieneusi comprising a forward primer comprising or consisting of at least 15 consecutive nucleotides of GAGTGTAGTATAGACTGGCGAA (SEQ ID NO:28) and a reverse primer comprising or consisting of at least 15 consecutive nucleotides of TCGTCCTTGATCCTAAGATACG (SEQ ID NO:29);
h) a primer pair for detecting Enterobius vermicularis comprising a forward primer comprising or consisting of at least 15 consecutive nucleotides of GCAGAGCTTTTCCAAAATTTATTTCC (SEQ ID NO:30) and a reverse primer comprising or consisting of at least 15 consecutive nucleotides of CCCAAGTTTGAGGTAATTTCTCG (SEQ ID NO:31);
i) a primer pair for detecting Diphyllobothrium latum and Diphyllobothrium nihonkaiense comprising a forward primer comprising or consisting of at least 15 consecutive nucleotides of CCAGTTATTACTGGTGTAAGATTGAA (SEQ ID NO:32) and a reverse primer comprising or consisting of at least 15 consecutive nucleotides of TCAAGCATAACCTGACTCATATAC (SEQ ID NO:33);
j) a primer pair for detecting Diphyllobothrium latum and Diphyllobothrium nihonkaiense comprising a forward primer comprising or consisting of at least 15 consecutive nucleotides of CCAGTTATTACTGGTGTAAGATTGAA (SEQ ID NO:34) and a reverse primer comprising or consisting of at least 15 consecutive nucleotides of TCAAGCATAACCTGACTCATATAC (SEQ ID NO:35);
k) a primer pair for detecting Diphyllobothrium latum and Diphyllobothrium nihonkaiense comprising a forward primer comprising or consisting of at least 15 consecutive nucleotides of CCAGTTATTACAGGTGTGAGATTG (SEQ ID NO:36) and a reverse primer comprising or consisting of at least 15 consecutive nucleotides of CAAGCATAACCCGACTCGTA (SEQ ID NO:37);
l) a primer pair for detecting Diphyllobothrium latum and Diphyllobothrium nihonkaiense comprising a forward primer comprising or consisting of at least 15 consecutive nucleotides of CCAGTTATTACAGGTGTGAGATTG (SEQ ID NO:36) and a reverse primer comprising or consisting of at least 15 consecutive nucleotides of CAAGCATAACCCGACTCGTA (SEQ ID NO:37);
m) a primer pair for detecting Schistosoma mansoni comprising a forward primer comprising or consisting of at least 15 consecutive nucleotides of AGGTGTTTTCATGACTTTATATGTTGA (SEQ ID NO:38) and a reverse primer comprising or consisting of at least 15 consecutive nucleotides of AGCAGATGCAGATAAAGCCA (SEQ ID NO:39);
n) a primer pair for detecting Blastocystis hominis comprising a forward primer comprising or consisting of at least 15 consecutive nucleotides of CAGCTTTCGATGGTAGTGTATTG (SEQ ID NO:40) and a reverse primer comprising or consisting of at least 15 consecutive nucleotides of GGCTCCCTCTCCGAAATC (SEQ ID NO:41);
o) a primer pair for detecting Blastocystis hominis comprising a forward primer comprising or consisting of at least 15 consecutive nucleotides of TCAGCTTTCGATGGTAGTATATGG (SEQ ID NO:42) and a reverse primer comprising or consisting of at least 15 consecutive nucleotides of GGCTCCCTCTCCGAAATC (SEQ ID NO:43);
p) a primer pair for detecting liver worms comprising a forward primer comprising or consisting of at least 15 consecutive nucleotides of AGCTCGTAGTTGGATCTGG (SEQ ID NO:44) and a reverse primer comprising or consisting of at least 15 consecutive nucleotides of CCACCAATCATGCTAACACC (SEQ ID NO:45);
q) a primer pair for detecting Ancylostoma duodenale comprising a forward primer comprising or consisting of at least 15 consecutive nucleotides of CAGTGTAGCTTGTGGCAC (SEQ ID NO:48) and a reverse primer comprising or consisting of at least 15 consecutive nucleotides of CAGCTAACGTACATGTTGCAATA (SEQ ID NO:49); and
r) a primer pair for detecting Ancylostoma duodenale comprising a forward primer comprising or consisting of at least 15 consecutive nucleotides of ACAGTGCAGCTTGTGGCA (SEQ ID NO:50) and a reverse primer comprising or consisting of at least 15 consecutive nucleotides of CAGCCAACGTACATGTTGCAATA (SEQ ID NO:51).

18. The composition according to claim 17, wherein the set of primer pairs comprises:
a) a primer pair for detecting Hymenolepis nana and Hymenolepis diminuta comprising a forward primer comprising or consisting of at least 15 consecutive nucleotides of AATTCCTGATGCTTTTGGGTTTTATG (SEQ ID NO:17) and a reverse primer comprising or consisting of at least 15 consecutive nucleotides of AGAACACTGCCGTCTTTACATCTAA (SEQ ID NO:18);
b) a primer pair for detecting Hymenolepis nana and Hymenolepis diminuta comprising a forward primer comprising or consisting of at least 15 consecutive nucleotides of AATTCCTGATGCTTTTGGGTTTTATG (SEQ ID NO:17) and a reverse primer comprising or consisting of at least 15 consecutive nucleotides of AAATACAGCCGTCTTAACATCCAA (SEQ ID NO:19); and
c) a primer pair for detecting liver worms comprising a forward primer comprising or consisting of at least 15 consecutive nucleotides of AGCTCGTAGTTGGATCTGG (SEQ ID NO:44) and a reverse primer comprising or consisting of at least 15 consecutive nucleotides of CCACCAATCATGCTAACACC (SEQ ID NO:45).

19. The composition according to claim 17, wherein the set of primer pairs comprises:
a) a primer pair for detecting Enterocytozoon bieneusi comprising a forward primer comprising or consisting of at least 15 consecutive nucleotides of GAGTGTAGTATAGACTGGCGAA (SEQ ID NO:28) and a reverse primer comprising or consisting of at least 15 consecutive nucleotides of TCGTCCTTGATCCTAAGATACG (SEQ ID NO:29);
b) a primer pair for detecting Enterobius vermicularis comprising a forward primer comprising or consisting of at least 15 consecutive nucleotides of GCAGAGCTTTTCCAAAATTTATTTCC (SEQ ID NO:30) and a reverse primer comprising or consisting of at least 15 consecutive nucleotides of CCCAAGTTTGAGGTAATTTCTCG (SEQ ID NO:31); and
c) a primer pair for detecting Schistosoma mansoni comprising a forward primer comprising or consisting of at least 15 consecutive nucleotides of AGGTGTTTTCATGACTTTATATGTTGA (SEQ ID NO:38) and a reverse primer comprising or consisting of at least 15 consecutive nucleotides of AGCAGATGCAGATAAAGCCA (SEQ ID NO:39).

20. The composition according to claim 17, wherein the probes comprise or consist of a sequence that is identical or complementary to at least 10 consecutive nucleotides of any of the probe sequences as set forth in SEQ ID NOS:52-67.

21. (canceled)

22. A kit for detecting the presence or absence of intestinal parasites in a sample, wherein said kit comprises a set of oligonucleotide primers and probes, wherein the probes each comprise a detectable label, and wherein the set of primers comprises one or more primer pairs selected from:
a) a primer pair for detecting Hymenolepis nana and Hymenolepis diminuta comprising a forward primer comprising or consisting of at least 15 consecutive nucleotides of AATTCCTGATGCTTTTGGGTTTTATG (SEQ ID NO:17) and a reverse primer comprising or consisting of at least 15 consecutive nucleotides of AGAACACTGCCGTCTTTACATCTAA (SEQ ID NO:18);
a primer pair for detecting Hymenolepis nana and Hymenolepis diminuta comprising a forward primer comprising or consisting of at least 15 consecutive nucleotides of AATTCCTGATGCTTTTGGGTTTTATG (SEQ ID NO:17) and a reverse primer comprising or consisting of at least 15 consecutive nucleotides of AAATACAGCCGTCTTAACATCCAA (SEQ ID NO:19);
c) a primer pair for detecting Fasciolopsis buski comprising a forward primer comprising or consisting of at least 15 consecutive nucleotides of CACTGTTCAAGTGGTATTGATTG (SEQ ID NO:20) and a reverse primer comprising or consisting of at least 15 consecutive nucleotides of CCAGGTTATCAGTCCTACCC (SEQ ID NO:21);
d) a primer pair for detecting E. intestinalis, E. cuniculi, and E. hellem comprising a forward primer comprising or consisting of at least 15 consecutive nucleotides of CTGAGTCCTGAGTGTTAGATAAGA (SEQ ID NO:22) and a reverse primer comprising or consisting of at least 15 consecutive nucleotides of CTAATGCCAATCAATCCCGTG (SEQ ID NO:23);
e) a primer pair for detecting E. intestinalis, E. cuniculi, and E. hellem comprising a forward primer comprising or consisting of at least 15 consecutive nucleotides of GTCCTTCGTGTTAGATAAGATATAAGTC (SEQ ID NO:24) and a reverse primer comprising or consisting of at least 15 consecutive nucleotides of AGATAATGCCAATCAATCCCATG (SEQ ID NO:25);
f) a primer pair for detecting E. intestinalis, E. cuniculi, and E. hellem comprising a forward primer comprising or consisting of at least 15 consecutive nucleotides of GACGAAGATTGAGAGGTCTGA (SEQ ID NO:26) and a reverse primer comprising or consisting of at least 15 consecutive nucleotides of CTAATGCCTATCAATCCCGTG (SEQ ID NO:27);
g) a primer pair for detecting Enterocytozoon bieneusi comprising a forward primer comprising or consisting of at least 15 consecutive nucleotides of GAGTGTAGTATAGACTGGCGAA (SEQ ID NO:28) and a reverse primer comprising or consisting of at least 15 consecutive nucleotides of TCGTCCTTGATCCTAAGATACG (SEQ ID NO:29);
h) a primer pair for detecting Enterobius vermicularis comprising a forward primer comprising or consisting of at least 15 consecutive nucleotides of GCAGAGCTTTTCCAAAATTTATTTCC (SEQ ID NO:30) and a reverse primer comprising or consisting of at least 15 consecutive nucleotides of CCCAAGTTTGAGGTAATTTCTCG (SEQ ID NO:31);
i) a primer pair for detecting Diphyllobothrium latum and Diphyllobothrium nihonkaiense comprising a forward primer comprising or consisting of at least 15 consecutive nucleotides of CCAGTTATTACTGGTGTAAGATTGAA (SEQ ID NO:32) and a reverse primer comprising or consisting of at least 15 consecutive nucleotides of TCAAGCATAACCTGACTCATATAC (SEQ ID NO:33);
j) a primer pair for detecting Diphyllobothrium latum and Diphyllobothrium nihonkaiense comprising a forward primer comprising or consisting of at least 15 consecutive nucleotides of CCAGTTATTACTGGTGTAAGATTGAA (SEQ ID NO:34) and a reverse primer comprising or consisting of at least 15 consecutive nucleotides of TCAAGCATAACCTGACTCATATAC (SEQ ID NO:35);
k) a primer pair for detecting Diphyllobothrium latum and Diphyllobothrium nihonkaiense comprising a forward primer comprising or consisting of at least 15 consecutive nucleotides of CCAGTTATTACAGGTGTGAGATTG (SEQ ID NO:36) and a reverse primer comprising or consisting of at least 15 consecutive nucleotides of CAAGCATAACCCGACTCGTA (SEQ ID NO:37);
l) a primer pair for detecting Diphyllobothrium latum and Diphyllobothrium nihonkaiense comprising a forward primer comprising or consisting of at least 15 consecutive nucleotides of CCAGTTATTACAGGTGTGAGATTG (SEQ ID NO:36) and a reverse primer comprising or consisting of at least 15 consecutive nucleotides of CAAGCATAACCCGACTCGTA (SEQ ID NO:37);
m) a primer pair for detecting Schistosoma mansoni comprising a forward primer comprising or consisting of at least 15 consecutive nucleotides of AGGTGTTTTCATGACTTTATATGTTGA (SEQ ID NO:38) and a reverse primer comprising or consisting of at least 15 consecutive nucleotides of AGCAGATGCAGATAAAGCCA (SEQ ID NO:39);
n) a primer pair for detecting Blastocystis hominis comprising a forward primer comprising or consisting of at least 15 consecutive nucleotides of CAGCTTTCGATGGTAGTGTATTG (SEQ ID NO:40) and a reverse primer comprising or consisting of at least 15 consecutive nucleotides of GGCTCCCTCTCCGAAATC (SEQ ID NO:41);
o) a primer pair for detecting Blastocystis hominis comprising a forward primer comprising or consisting of at least 15 consecutive nucleotides of TCAGCTTTCGATGGTAGTATATGG (SEQ ID NO:42) and a reverse primer comprising or consisting of at least 15 consecutive nucleotides of GGCTCCCTCTCCGAAATC (SEQ ID NO:43);
p) a primer pair for detecting liver worms comprising a forward primer comprising or consisting of at least 15 consecutive nucleotides of AGCTCGTAGTTGGATCTGG (SEQ ID NO:44) and a reverse primer comprising or consisting of at least 15 consecutive nucleotides of CCACCAATCATGCTAACACC (SEQ ID NO:45);
q) a primer pair for detecting Ancylostoma duodenale comprising a forward primer comprising or consisting of at least 15 consecutive nucleotides of CAGTGTAGCTTGTGGCAC (SEQ ID NO:48) and a reverse primer comprising or consisting of at least 15 consecutive nucleotides of CAGCTAACGTACATGTTGCAATA (SEQ ID NO:49); and
r) a primer pair for detecting Ancylostoma duodenale comprising a forward primer comprising or consisting of at least 15 consecutive nucleotides of ACAGTGCAGCTTGTGGCA (SEQ ID NO:50) and a reverse primer comprising or consisting of at least 15 consecutive nucleotides of CAGCCAACGTACATGTTGCAATA (SEQ ID NO:51).

23. The kit according to claim 22, comprising other PCR reagent components selected from the group consisting of: a polymerase, nucleotides, buffer, salts, detergents and/or other additives.

24. The kit according to claim 22, further comprising one or more control primers, additional probes or nucleotide sequences.

25. The kit according to claim 22, wherein the one or more probes comprise or consist of a sequence that is identical or complementary to at least 10 consecutive nucleotides of any of the probe sequences as set forth in SEQ ID NOS:52-67.

26. The method according to claim 1, wherein the method comprises detecting the presence or absence of one or more intestinal parasites and optionally one or more controls in a multiplex real-time PCR assay.


SEQUENCE LISTING (67 sequences)

SEQ ID NO: 1 - DNA, 125 bp, Unknown
  Hymenolepis sp.
aattcctgatgcttttgggttttatggtttattatttgctatgttttctatagtgtgcttaggttgtagtgtgtgggctcatcatatgtttactgttggtttggatgttaagacggctgtatttt

SEQ ID NO: 2 - DNA, 125 bp, Unknown
  Hymenolepis sp.
aattcctgatgcttttgggttttatgggctcttgtttgccatgttttctattgtttgtttaggtagaagtgtttgagggcatcatatgtttactgttggtttagatgtaaagacggcagtgttct

SEQ ID NO: 3 - DNA, 129 bp, Fasciolopsis buski
cactgttcaagtggtattgattgggttcgcccattctttgccattgccctcgcatgcacctggtccttgtggccggactgcacgtacgtcgcccggcggtgcctatcccgggtaggactgataacctgg

SEQ ID NO: 4 - DNA, 164 bp, Unknown
  Encephalitozoon sp
gacgaagatcggaaggtctgagtcctgagtgttagataagatataagtcgtaacatggctgctgttggagaaccagcagcaggatcagtatgttgttgtgttttgatggatgtttgtttgtttgtttgtggtttctctgttcacgggattgattggcattagcg

SEQ ID NO: 5 - DNA, 151 bp, Unknown
  Encephalitozoon sp
gacgaagattggaaggtctgagtccttcgtgttagataagatataagtcgtaacgcggctgctgttggagaaccagcagcaggatcagtatttgagagattggggggaatttttttgatttgaggatccacgggattgataggcattagca

SEQ ID NO: 6 - DNA, 169 bp, Unknown
  Encephalitozoon sp
gacgaagattgagaggtctgagtctttcgtgttagataagatataagtcgtaacatggctgctgttggagaaccagcagcaggatcagtatgttgatttgattgatttgtggggatttttagttttttagtttttctttctctatccatgggattgattggcattatct

SEQ ID NO: 7 - DNA, 100 bp, Enterocytozoon bieneusi
gagtgtagtatagactggcgaagaatgaaatctcaagacccagtttggactaacggaggcgaaggcgacactcttagacgtatcttaggatcaaggacga

SEQ ID NO: 8 - DNA, 87 bp, Enterobius vermicularis
gcagagcttttccaaaatttatttccaagccacagactcactgatgttcatgtctgagccggaacgagaaattacctcaaacttggg

SEQ ID NO: 9 - DNA, 156 bp, Unknown
  Diphyllobothrium latum/nihonkaiense
ccagttattacaggtgtgagattgaataagtatttattacaatgtcattgtatagtttctaatgttggtttcaatttatgttttttccctatgcattactttggtgtgtgcggtttaccacgtcgtgtgtgtgtgtacgagtcgggttatgcttga

SEQ ID NO: 10 - DNA, 156 bp, Unknown
  Diphyllobothrium latum/nihonkaiense
ccagttattactggtgtaagattgaataagtatttactacaatgtcattgtatagtttctaatgttggtttcaatttatgtttttttcccatgcattattttggtgtgtgcggtttaccacgtcgtgtgtgcgtatatgagtcaggttatgcttga

SEQ ID NO: 11 - DNA, 156 bp, Unknown
  Diphyllobothrium latum/nihonkaiense
ccagttattactggtgtaagattgaataagtatttactacaatgtcattgtatagtttctaatgttggtttcaatttatgtttttttcctatgcattattttggtgtgtgcggtttaccacgtcgtgtgtgtgtatatgagtcaggttatgcttga

SEQ ID NO: 12 - DNA, 156 bp, Unknown
  Diphyllobothrium latum/nihonkaiense
ccagttattactggtgtgagattgaataagtatttactacaatgtcattgtatagtttctaatgttggtttcaatttatgtttttttcctatgcattattttggtgtgtgcggtttaccacgtcgtgtgtgcgtatatgagtcaggttatgcttga

SEQ ID NO: 13 - DNA, 134 bp, Schistosoma mansoni
aggtgttttcatgactttatatgttgaatagttgcggtatgcgggttttagatcccatagtatggtgattagtcggttttatatttttatttacggttggtggtgtcacaggggtggctttatctgcatctgct

SEQ ID NO: 14 - DNA, 82 bp, Blastocystis hominis
tcagctttcgatggtagtgtattggactaccatggcagtaacgggtaacgaagaatttgggttcgatttcggagagggagcc

SEQ ID NO: 15 - DNA, 82 bp, Blastocystis hominis
tcagctttcgatggtagtatatgggcctaccatggcagtaacgggtaacgaagaatttgggttcgatttcggagagggagcc

SEQ ID NO: 16 - DNA, 95 bp, Unknown
  C. sinensis / Opistothorchis sp. / Metorchis sp.
agctcgtagttggatctgggtcgcatggctacatgccgttgctcgtattcctggcctggttcacaccgggacgggtttgtgagtcggtgtcgtgg

SEQ ID NO: 17 - DNA, 26 bp, Unknown
  Hymenolepis sp.
aattcctgatgcttttgggttttatg

SEQ ID NO: 18 - DNA, 25 bp, Unknown
  Hymenolepis sp.
agaacactgccgtctttacatctaa

SEQ ID NO: 19 - DNA, 24 bp, Unknown
  Hymenolepis sp.
aaatacagccgtcttaacatccaa

SEQ ID NO: 20 - DNA, 23 bp, Fasciolopsis buski
cactgttcaagtggtattgattg

SEQ ID NO: 21 - DNA, 20 bp, Fasciolopsis buski
ccaggttatcagtcctaccc

SEQ ID NO: 22 - DNA, 24 bp, Unknown
  Encephalitozoon sp
ctgagtcctgagtgttagataaga

SEQ ID NO: 23 - DNA, 21 bp, Unknown
  Encephalitozoon sp
ctaatgccaatcaatcccgtg

SEQ ID NO: 24 - DNA, 28 bp, Unknown
  Encephalitozoon sp
gtccttcgtgttagataagatataagtc

SEQ ID NO: 25 - DNA, 23 bp, Unknown
  Encephalitozoon sp
agataatgccaatcaatcccatg

SEQ ID NO: 26 - DNA, 21 bp, Unknown
  Encephalitozoon sp
gacgaagattgagaggtctga

SEQ ID NO: 27 - DNA, 21 bp, Unknown
  Encephalitozoon sp
ctaatgcctatcaatcccgtg

SEQ ID NO: 28 - DNA, 22 bp, Enterocytozoon bieneusi
gagtgtagtatagactggcgaa

SEQ ID NO: 29 - DNA, 22 bp, Enterocytozoon bieneusi
tcgtccttgatcctaagatacg

SEQ ID NO: 30 - DNA, 26 bp, Enterobius vermicularis
gcagagcttttccaaaatttatttcc

SEQ ID NO: 31 - DNA, 23 bp, Enterobius vermicularis
cccaagtttgaggtaatttctcg

SEQ ID NO: 32 - DNA, 26 bp, Unknown
  Diphyllobothrium latum/nihonkaiense
ccagttattactggtgtaagattgaa

SEQ ID NO: 33 - DNA, 24 bp, Unknown
  Diphyllobothrium latum/nihonkaiense
tcaagcataacctgactcatatac

SEQ ID NO: 34 - DNA, 26 bp, Unknown
  Diphyllobothrium latum/nihonkaiense
ccagttattactggtgtaagattgaa

SEQ ID NO: 35 - DNA, 24 bp, Unknown
  Diphyllobothrium latum/nihonkaiense
tcaagcataacctgactcatatac

SEQ ID NO: 36 - DNA, 24 bp, Unknown
  Diphyllobothrium latum/nihonkaiense
ccagttattacaggtgtgagattg

SEQ ID NO: 37 - DNA, 20 bp, Unknown
  Diphyllobothrium latum/nihonkaiense
caagcataacccgactcgta

SEQ ID NO: 38 - DNA, 27 bp, Schistosoma mansoni
aggtgttttcatgactttatatgttga

SEQ ID NO: 39 - DNA, 20 bp, Schistosoma mansoni
agcagatgcagataaagcca

SEQ ID NO: 40 - DNA, 23 bp, Blastocystis hominis
cagctttcgatggtagtgtattg

SEQ ID NO: 41 - DNA, 18 bp, Blastocystis hominis
ggctccctctccgaaatc

SEQ ID NO: 42 - DNA, 24 bp, Blastocystis hominis
tcagctttcgatggtagtatatgg

SEQ ID NO: 43 - DNA, 18 bp, Blastocystis hominis
ggctccctctccgaaatc

SEQ ID NO: 44 - DNA, 19 bp, Unknown
  C. sinensis / Opistothorchis sp. / Metorchis sp.
agctcgtagttggatctgg

SEQ ID NO: 45 - DNA, 20 bp, Unknown
  C. sinensis / Opistothorchis sp. / Metorchis sp.
ccaccaatcatgctaacacc

SEQ ID NO: 46 - DNA, 259 bp, Ancylostoma duodenale
cccatgagacatacaaaaaggtaatgccgccgtctggttcagggttgtttatatctactacagtgtagcttgtggcactgtttgtcgaacggcacttgcttttagcgattcccgttctagatcagaatatattgcaacatgtacgttagctggctagtttgctaacgtgcgctgaatgacagcaaactcgttgttgctgctgaatcgttcaccgactttagaacgtttcgggtctcgactatacgcccgttttcggatc

SEQ ID NO: 47 - DNA, 241 bp, Ancylostoma duodenale
cccatgagacatacaaaaaggtaatgccgcctatatctactacagtgcagcttgtggcactgtttgtcgaacggcacttgcttttagcgattcccgttctagatcagaatatattgcaacatgtacgttggctggctagtttgctaacgtgcgctgaatgacagcaaactcgttgttgctgctgaatcgtttaccgactttagaacgtttcgggtctcgactatacgcccgttttcggatc

SEQ ID NO: 48 - DNA, 18 bp, Ancylostoma duodenale
cagtgtagcttgtggcac

SEQ ID NO: 49 - DNA, 23 bp, Ancylostoma duodenale
cagctaacgtacatgttgcaata

SEQ ID NO: 50 - DNA, 18 bp, Ancylostoma duodenale
acagtgcagcttgtggca

SEQ ID NO: 51 - DNA, 23 bp, Ancylostoma duodenale
cagccaacgtacatgttgcaata

SEQ ID NO: 52 - DNA, 32 bp, Hymenolepis nana
agtgtgcttaggttgtagtgtgtgggctcatc

SEQ ID NO: 53 - DNA, 31 bp, Hymenolepis diminuta
tgtttgccatgttttctattgtttgtttagg

SEQ ID NO: 54 - DNA, 24 bp, Fasciolopsis buski
ttcgcccattctttgccattgccc

SEQ ID NO: 55 - DNA, 27 bp, Encephalitozoon intestinalis
ctgatcctgctgctggttctccaacag

SEQ ID NO: 56 - DNA, 29 bp, Encephalitozoon cuniculi
atgatcctgctaatggttctccaacagca

SEQ ID NO: 57 - DNA, 29 bp, Encephalitozoon hellem
atgatcctgctaatggttctccaacagca

SEQ ID NO: 58 - DNA, 23 bp, Enterocytozoon bieneusi
agtgtcgccttcgcctccgttag

SEQ ID NO: 59 - DNA, 30 bp, Enterobius vermicularis
tccggctcagacatgaacatcagtgagtct

SEQ ID NO: 60 - DNA, 24 bp, Diphyllobothrium latum
acacgacgtggtaaaccgcacaca

SEQ ID NO: 61 - DNA, 24 bp, Diphyllobothrium latum
acacgacgtggtaaaccgcacaca

SEQ ID NO: 62 - DNA, 24 bp, Diphyllobothrium latum
acacgacgtggtaaaccgcacaca

SEQ ID NO: 63 - DNA, 24 bp, Diphyllobothrium nihonkaiense
acacgacgtggtaaaccgcacaca

SEQ ID NO: 64 - DNA, 22 bp, Schistosoma mansoni
cccctgtgacaccaccaaccgt

SEQ ID NO: 65 - DNA, 30 bp, Blastocystis hominis
aaattcttcgttacccgttactgccatggt

SEQ ID NO: 66 - DNA, 30 bp, Blastocystis hominis; identical to SEQ ID NO: 65

SEQ ID NO: 67 - DNA, 25 bp, Clonorchis sinensis
ttgctcgtattcctggcctggttca